(19) United States
(12) Patent Application Publication
(10) Pub. No.: US 2022/0031708 A1
(43) Pub. Date: Feb. 3, 2022

(54) METHOD FOR TREATING IDIOPATHIC PULMONARY FIBROSIS

(71) Applicant: NuMedii, Inc., San Mateo, CA (US)

(72) Inventors: Asim Siddiqui, San Francisco, CA (US); Heather Arnett, Seattle, WA (US); Linda De Young, Montara, CA (US); Tarangini Deshpande, Menlo Park, CA (US)

(21) Appl. No.: 17/376,311

(22) Filed: Jul. 15, 2021

Related U.S. Application Data
(63) Continuation of application No. PCT/US20/14973, filed on Jan. 24, 2020.
(60) Provisional application No. 62/796,964, filed on Jan. 25, 2019.

(51) Int. Cl. A61K 31/5377; A61K 31/496; A61K 31/4418; C12N 5/071; A61P 11/00

(52) U.S. Cl. CPC A61K 31/5377; A61K 31/496; C12N 2501/727; C12N 5/0688; A61P 11/00; A61K 31/4418

(57) ABSTRACT

Provided is a method of treating idiopathic pulmonary fibrosis (IPF) using an agent that reduces or eliminates the kinase activity of checkpoint kinase 1 (Chk1).

### CROSS REFERENCE

This application is a continuation of International Patent Application No. PCT/US20/14973, filed Jan. 24, 2020, which claims the benefit of U.S. Provisional Patent Application No. 62/796,964, filed Jan. 25, 2019, each which is incorporated herein by reference in its entirety.

### BACKGROUND

Idiopathic Pulmonary Fibrosis (IPF) is a specific form of chronic, progressive interstitial lung disease that is characterized by the presence of excessive scar tissue (i.e., fibrosis) in the pulmonary interstitium. IPF is observed as a histological pattern of usual interstitial pneumonia (UIP). Patients undergo a progressive worsening of dyspnea and lung function, and have a poor prognosis, with 50% mortality within 3-5 years after diagnosis. IPF is estimated to afflict about 1 out of 200 adults of age 60 and over in the U.S., which translates to more than 200,000 people living with IPF in the U.S. today. Approximately 50,000 new cases are diagnosed each year, and as many as 40,000 Americans die from IPF each year. Males are affected approximately twice as often as females.

### SUMMARY

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of an activation marker in the subject by at least about 5% relative to a control.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces a level of fibroblast to myofibroblast differentiation in the subject by at least about 5% relative to a control.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces collagen deposition in a lung of the subject by at least about 5% relative to a control subject that was not administered the Chk1 inhibitor.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of a cytokine in a lung of the subject by at least about 5% relative to a control.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of a pro-fibrotic mediator in a lung of the subject by at least about 5% relative to a control.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces epithelial cell expression of a senescence-associated gene in a lung of the subject by at least about 5% relative to a control.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of an activation marker in the subject by at least about 5%, reduces a level of fibroblast to myofibroblast differentiation in the subject by at least about 5%, reduces collagen deposition in a lung of the subject by at least about 5%, reduces macrophage expression of a cytokine in the lung of the subject by at least about 5%, reduces macrophage expression of a pro-fibrotic mediator in the lung of the subject by at least about 5%, and reduces epithelial cell expression of a senescence-associated gene in the lung of the subject by at least about 5% relative to a control, wherein the control is a control subject that was not administered the Chk1 inhibitor.

Disclosed herein is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein the Chk1 inhibitor is not a thiazole compound, a heterocyclic urea compound, a heterocyclic thiourea compound, or an anilinopiperazine compound. In some embodiments, the Chk1 inhibitor is not a thiazole compound, a heterocyclic urea compound, a heterocyclic thiourea compound, or an anilinopiperazine compound.

Disclosed herein is a method of reducing activation of a macrophage, comprising contacting a population of cells with a Chk1 inhibitor, wherein the population of cells comprises the macrophage, wherein upon contacting the population of cells with the Chk1 inhibitor, the macrophage exhibits an expression level of an activation marker that is at least about 5% lower than an expression level of the activation marker by a macrophage that was not contacted with the Chk1 inhibitor.

Disclosed herein is a method of reducing differentiation of a fibroblast into a myofibroblast, comprising contacting a population of cells with a Chk1 inhibitor, wherein the population of cells comprises the fibroblast, wherein upon contacting the population of cells with the Chk1 inhibitor, the fibroblast exhibits an expression level of alpha smooth muscle actin that is at least about 5% lower than an expression level of the alpha smooth muscle actin by a fibroblast that was not contacted with the Chk1 inhibitor.

Disclosed herein is a method of reducing collagen deposition, comprising contacting a tissue with a Chk1 inhibitor, wherein upon contacting the tissue with the Chk1 inhibitor, the tissue exhibits an at least about 5% lower level of an indicator of collagen deposition relative to a tissue that was not contacted with the Chk1 inhibitor.

Disclosed herein is a method of reducing a level of a cytokine, comprising contacting a population of cells with a Chk1 inhibitor, wherein the population of cells comprises a macrophage, wherein upon contacting the population of cells with the Chk1 inhibitor, the macrophage produces a level of the cytokine that is at least about 5% lower than a level of the cytokine produced by a macrophage that was not contacted with the Chk1 inhibitor.

Disclosed herein is a method of reducing a level of a pro-fibrotic mediator, comprising contacting a population of cells with a Chk1 inhibitor, wherein the population of cells comprises a macrophage, wherein upon contacting the population of cells with the Chk1 inhibitor, the macrophage produces a level of the pro-fibrotic mediator that is at least about 5% lower than a level of the pro-fibrotic mediator produced by a macrophage that was not contacted with the Chk1 inhibitor.

Disclosed herein is a method of reducing cellular senescence, comprising contacting a population of cells with a Chk1 inhibitor, wherein the population of cells comprises an epithelial cell, wherein upon contacting the population of cells with the Chk1 inhibitor, the epithelial cell expresses a level of a senescence-associated gene that is at least about 5% lower than a level of the senescence-associated gene expressed by an epithelial cell that was not contacted with the Chk1 inhibitor.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIGS. 1A and 1B show that Chk1 inhibition blocks differentiation of lung fibroblasts isolated from human IPF donor lungs into myofibroblasts. Human lung fibroblasts from 3 donors were seeded in 96-well plates and treated with 1.25 ng/mL TGFb to induce differentiation to a myofibroblast phenotype (characterized by the induction of alpha smooth muscle actin [aSMA]). Chk1 inhibitors PF-477736 (FIG. 1A) or rabusertib (FIG. 1B) were dosed as indicated in an 8-point concentration curve 1 hour prior to cytokine treatment. Staining for aSMA and DAPI was assessed after 72 hours using high content imaging analysis, and is represented as percent inhibition of aSMA induction and % viable cells.

FIGS. 2A-2C demonstrate that Chk1 inhibition impacts macrophage activation to M1 and M2 phenotypes, and alters the production of pro-inflammatory cytokines and pro-fibrotic mediators. Human monocytes were isolated from peripheral blood and differentiated into M1 macrophages by treatment with LPS/IFNg, or M2 macrophages by treatment with IL-4, for 3 days. Chk1 inhibitor PF-477736 was dosed as indicated 1 hr prior to cytokine treatment. Flow cytometry was performed for CD80 as a marker of M1 differentiation after LPS/IFNg treatment (FIG. 2A), and CD163 as a marker of M2 differentiation after IL-4 treatment (FIG. 2B). M1-polarized cell supernatants were tested in a Luminex-based assay for 51 analytes (cytokines, MMPs). Representative changes in some analytes are shown (FIG. 2C).

FIG. 3A-3C demonstrate that Chk1 inhibition decreases histopathology and collagen deposition a mouse model of pulmonary fibrosis. FIG. 3A shows that PF-477736 and nintedanib improved histopathology scores in the mouse model of pulmonary fibrosis. FIG. 3B provides histograms for each study arm. FIG. 3B provides empirical cumulative density function (ECDF) curves demonstrating that PF-477736 treatment resulted in a greater reduction in pathology severity compared to nintedanib.

FIGS. 4A and 4B demonstrate collagen deposition in animals treated with PF-477736 (FIG. 4A) or nintedanib (FIG. 4B).

FIGS. 5A and 5B demonstrate that Chk1 activity is found in IPF-specific populations of epithelial cells, fibroblasts and macrophages. FIG. 5A demonstrates clustering of IPF-specific epithelial, macrophage, and fibroblast cell populations from single cell RNA seq data of IPF, healthy control, and COPD lungs. FIG. 5B demonstrates increased expression of genes that correlate with Chk1 activity in IPF-specific cell populations.

FIG. 6 shows that the concentration of Chk1 inhibitor required for 50% inhibition of fibroblast to myofibroblast differentiation was lower when co-administered with nintedanib.

FIG. 7 shows that the concentration of nintedanib required for 50% inhibition of fibroblast to myofibroblast differentiation was lower when co-administered with a Chk1 inhibitor

FIGS. 8A-8F show that high Chk1 activity correlates with expression of senescence-associated secreted proteins in epithelial cells. FIG. 8A demonstrates epithelial cell subsets identified in a single cell RNA sequencing dataset, including alveolar type I (AT-I) epithelial cells, alveolar type II (AT-II) epithelial cells, basal epithelial cells, ciliated epithelial cells, club epithelial cells, goblet cells, and an IPF-associated epithelial cell subset. FIG. 8B shows cells identified as being from IPF patients and healthy controls. FIG. 8C summarizes expression of senescence-associated genes. FIG. 8D summarizes a Chk1 activity as indicated by a signature of 100 Chk1-correlated genes. FIG. 8E shows that a threshold of approximately 0.1 to 0.2 is appropriate for differentiating between Chk1 high and Chk1 low cells. FIG. 8F shows that the Chk1 high group expresses higher mean levels of senescence-associated secretory proteins when a threshold of 0.1 to 0.2 is applied, and that in general, cells with high Chk1 activity have higher expression of senescence associated genes.

FIG. 9 illustrates the effects of GDC-0575 on fibroblast-to-myofibroblast differentiation.

FIG. 10 illustrates the effects of MK-8776 on fibroblast-to-myofibroblast differentiation.

FIG. 11 illustrates the effects of CCT-245737 on fibroblast-to-myofibroblast differentiation.

FIG. 12 illustrates the effects of BML-277 on fibroblast-to-myofibroblast differentiation.

FIG. 13 illustrates the effects of AZD-7762 on fibroblast-to-myofibroblast differentiation.

FIG. 14 illustrates the effects of prexasertib on fibroblast-to-myofibroblast differentiation.

FIG. 15 illustrates the effects of CCT-241533 on fibroblast-to-myofibroblast differentiation.

FIG. 16 provides control analysis of IPF donor samples dosed with control compounds.

FIG. 17 provides control analysis of healthy controls dosed with control compounds.

### DETAILED DESCRIPTION OF THE INVENTION

There are more than 100 different interstitial lung diseases (ILDs), including pulmonary fibrosis (PF). The treatment and outlook for each can vary significantly (see, for example, Meyer K C. Diagnosis and management of interstitial lung disease. Transl Respir Med. 2014; 2:4.), suggesting that the processes underlying these ILDs can be quite diverse. For instance, standard-of-care treatment for many ILDs include corticosteroids whereas the use of corticosteroids and immunosuppressive agents can be strongly discouraged in IPF and can worsen progression of IPF (see, for example, Raghu G et al. An official ATS/ERS/JRS/ALAT statement: idiopathic pulmonary fibrosis: evidence-based guidelines for diagnosis and management. Am J Respir Crit Care Med. 2011; 183(6):788-824). The distinctive processes underlying IPF can also be evident in comparisons of large-scale transcriptional analyses of fibrotic diseases (e.g. transcriptome microarrays, RNAseq, etc.): although some overlap in regulated pathways can be found in transcriptomes, most gene regulation appears to be disease-specific (see, for example, Makarev E et al. Common pathway signature in lung and liver fibrosis. Cell Cycle. 2016; 15(13):1667-73; Mahoney J M et al. Systems level analysis of systemic sclerosis shows a network of immune and profibrotic pathways connected with genetic polymorphisms. PLoS Comput Biol. 2015; 11(1):e1004005; Xu Y et al. Single-cell RNA sequencing identifies diverse roles of epithelial cells in idiopathic pulmonary fibrosis. JCI Insight. 2016; 1(20):e90558).

Although the mechanistic underpinnings of IPF are unclear, without wishing to be bound by theory, a current hypothesis is that the accumulated scar tissue in IPF stems from a wound healing process that fails to adequately resolve.

Wound healing can normally serve to 1) reduce blood loss via immediate clotting, and 2) re-establish an epithelial barrier by restoring tissue architecture.

Accordingly, in the first steps of wound healing, platelets can be recruited to the site of injury to temporarily plug damaged blood vessels. In addition, the activities of enzymes that degrade constituents of the extracellular matrix (ECM) can be up-regulated, and thereby increase the permeability of the tissue for white blood cells (e.g., monocytes, macrophages) from the surrounding capillaries and tissue and the clearance of cell debris from the tissue (e.g., by recruited phagocytic macrophages).

In the next steps of wound healing, the recruited white blood cells can secrete inflammatory cytokines. The mounted inflammatory response can stimulate fibroblasts to migrate to the site of injury and to differentiate into highly contractile and proliferative myofibroblasts that secrete new ECM proteins (e.g., collagen, fibronectins, proteoglycans, elastin).

As wound healing progresses, the process can first be slowed, and then ended. Such slowing and ending can involve a shift from ECM protein deposition to no net increase in ECM protein deposition. Under normal circumstances, this shift can include removal of myofibroblasts via apoptosis or phagocytic macrophages, and reduction of inflammation.

In IPF, however, the myofibroblasts can remain in the pulmonary tissue and continue to produce ECM proteins, leading to formation of scar tissue (i.e., progressive fibrosis). Furthermore, because myofibroblasts are contractile, they can pull the ECM into tight bundles, giving the tissue a higher tensile strength. In a tissue such as the lung, which requires constant movement to do its job (i.e., respiration), excess scar tissue can result in progressive impairment of the diffusing capacity for carbon monoxide and oxygen. Early symptoms of IPF can include shortness of breath and cough, which can gradually progress and end in death.

Without wishing to be bound by theory, persistence of the myofibroblasts in IPF appears to involve a propagation of the wound healing process and ongoing chronic inflammation through a variety of mechanisms, including the production of pro-inflammatory and pro-fibrotic mediators (e.g., TGFb, cytokines, growth factors) by both resident tissue macrophages and monocyte-derived macrophages from the blood. Although the precise phenotype of macrophages in the IPF lung is debated, roles for both M1 and M2 macrophages in promoting the disease have been proposed. The dysregulated inflammation in turn can continue to induce fibroblasts to differentiate into myofibroblasts. Impacting the formation of myofibroblasts (e.g., by inhibiting activation of macrophages, production of cytokines by macrophages, and/or reducing differentiation of fibroblasts into myofibroblasts), myofibroblasts survival, myofibroblasts contractility, or the ability of myofibroblasts to produce extracellular matrix, are all predicted to decrease the progressive nature of fibrosis, and potentially to decrease the amount of existing fibrosis. In some embodiments, the disclosure provides therapies that impact myofibroblast differentiation and function for the treatment of IPF. In some embodiments, the disclosure provides therapies that impact macrophage activation, macrophage differentiation, and/or macrophage function (e.g., production of cytokines and/or pro-fibrotic mediators by macrophages).

Two approved drugs for IPF are pirfenidone and nintedanib. Both have been proposed to impact fibroblast-to-myofibroblast transition, through mechanisms that are not entirely understood. However, of note, these drugs can exhibit only modest impacts both on the in vitro biology and in the clinic. Lung transplantation is considered a definitive therapy for IPF, but the 5-year survival after lung transplantation is less than 50%, and there are far fewer lungs available than there are patients in need. In some embodiments, compositions and methods of the disclosure provide a larger therapeutic benefit for IPF patients than existing treatments.

Without wishing to be bound by theory, cellular senescence can also contribute to the pathogenesis of IPF. For example, higher expression of senescence-associated genes and/or senescence-associated secretory proteins, e.g., by epithelial cells in the lungs, can contribute to IPF pathogenesis. In some embodiments, the disclosure provides therapies that impact cellular senescence for the treatment of IPF.

In some embodiments, the disclosure provides administering a treatment to a subject in need thereof, e.g., a subject needing treatment for IPF. Non-limiting examples of subjects include a human or non-human vertebrate (e.g., a mammal [e.g., human, non-human primate (e.g., monkey, orangutan, chimpanzee), domesticated animal (e.g., equine [e.g., horse], bovine [e.g., cattle], porcine [e.g., pig], ovine [e.g., sheep], rodent [e.g., mouse, rat, hamster, guinea pig], canine [e.g., dog], feline [e.g., cat], lagomorph [e.g., rabbit], caprine [e.g., goat]). In some embodiments, a subject can be at risk of (e.g., susceptible to) developing IPF or can have IPF.

“Treating” can refer to the mitigation of a disease condition. Non-limiting examples of treating can include: preventing the disease condition from occurring (e.g., in a subject predisposed to the disease condition, prior to manifestation of symptoms characteristic of the disease condition), modulating (i.e., reducing, ameliorating, inhibiting, or delaying further progression of) the disease condition, healing (i.e., eradicating) the disease condition (e.g., healing fibrotic wounds), increasing survival time of a subject with IPF, and reducing the risk of death in a subject with IPF.

### Method of Treating Idiopathic Pulmonary Fibrosis

In one aspect, provided herein is a method of treating idiopathic pulmonary fibrosis (IPF), wherein the method comprises the step of administering to a subject needing treatment for IPF a therapeutically effective amount of an agent that reduces or eliminates the kinase activity of checkpoint kinase 1 (Chk1).

In some embodiments, the method provided herein is effective at reducing at least one pathology in the pulmonary tissue of the subject selected from the group consisting of:
an aberrant (i.e., different compared to a healthy subject) rate of proliferation of fibroblasts;
an aberrant rate of differentiation of fibroblasts into myofibroblasts;
an aberrant formation of fibrotic loci (i.e., loci of rapid myofibroblast proliferation);
an aberrant deposition of ECM protein (e.g., collagen, fibronectin, elastin, and/or proteoglycans);
an aberrant myofibroblast contractile activity;
an aberrant rate of myofibroblast apoptosis;
an aberrant attachment of myofibroblasts to an ECM component;
an aberrant activation of macrophages;
an aberrant production of cytokines (e.g., TGFb, cytokines, growth factors) by macrophages;
an aberrant inflammation;
an aberrant growth of scar tissue;
an aberrant expression of senescence-associated genes.

Chk1 inhibition can lead to normalization of the gene expression profile in IPF. Chk1 is a protein kinase that can play an important role as a checkpoint in cell cycle progression. By analyzing the transcriptome of pulmonary fibroblasts of IPF patients and identifying cell perturbation signatures, the inventors identified inhibition of the kinase activity of Chk1 as a target for IPF. Notably, although fibrosis is a key pathological feature of many diseases, including IPF, Scleroderma, COPD, keloids, myelofibrosis, ulcerative colitis, uterine fibroids, and cardiac fibrosis, the bioinformatics prediction that Chk1 inhibition has the ability reverse a disease phenotype can be specific to IPF. The inventors have furthermore established that agents that reduce or eliminate the kinase activity of Chk1 can inhibit differentiation of human fibroblasts into myofibroblasts, reduce activation of macrophages towards an M1 or M2 phenotype, and reduce production of a number of pro-fibrotic and pro-inflammatory mediators.

Advantages of the method provided herein include superior treatment outcomes (e.g., increased pulmonary function, slower decrease in lung function over time, decreased pulmonary fibrosis, longer time until lung transplant, longer median survival time, increased quality of life measurements).

Agent that Reduces or Eliminates Kinase Activity of Chk1

The agent to be administered in the method provided herein can be any pharmaceutically acceptable and pharmaceutically active compound, a prodrug, or a pharmaceutically acceptable salt or ester thereof that reduces or eliminates the kinase activity of Chk1.

Agents that exhibit high therapeutic indices (i.e., high dose ratios between toxic and therapeutic effects [e.g., ratio of maximum tolerated dose [MTD] and ED50 [i.e., effective dose for 50% maximal response]) can be preferred.

In some embodiments, the agent reduces the kinase activity of Chk1 by at least 5% or at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 96%, at least 97%, at least 98%, or at least 99%.

In some embodiments, the agent has an IC50 (i.e., concentration for 50% inhibition) or an EC50 (i.e., concentration for 50% effect) for Chk1 in a range of 100 μM or less, 70 μM or less, 50 μM or less, 25 μM or less, 20 μM or less, 15 μM or less, 10 μM or less, 5 μM or less, 1 μM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 1 nM or less, 500 pM or less, or 100 pM or less.

In some embodiments, the agent reduces or eliminates the kinase activity of Chk1 only. In some embodiments, the agent reduces or eliminates the kinase activity of Chk1 as well as the kinase activity of one or more additional kinases. Non-limiting examples of such other kinases include Chk2; Foxo1; and AurKA, B, and C. In some embodiments, the agent reduces or eliminates the kinase activity of Chk1 specifically, for example, exhibits Chk1 inhibition at a lower concentration than is required for inhibition of other kinases.

In some embodiments, the agent reduces the kinase activity of Chk1 by at least 50%, at least 60%, at least 70%, at least 80%, at least 85%, at least 90%, or at least 95%, and reduces the kinase activity of one or more other kinases by less than 80%, less than 70%, less than 60%, less than 50%, less than 40%, less than 25%, less than 15%, less than 10%, less than 5%, or less than 1%.

In some embodiments, the agent reduces or eliminates the kinase activity of Chk1 and increases the kinase activity of another kinase.

Non-limiting examples of suitable compounds include AB-IsoG (isogranulatimide); AZD-7762; CCT-244747; CHK1-A; GNE-900; MK-8776; PF-477736; rabusertib; GDC-0425; GDC-0575; SAR 020106; V-158411; XL-844; ARRY 575; CASC-578; LY-2880070; CCT-245737; CCT-241533; prexasertib; VER-250840; and BML-277.

In some embodiments, the compound is PF-477736. In some embodiments, the compound is rabusertib. In some embodiments, the compound is AB-IsoG (isogranulatimide). In some embodiments, the compound is AZD-7762. In some embodiments, the compound is CCT-244747. In some embodiments, the compound is CHK1-A. In some embodiments, the compound is GNE-900. In some embodiments, the compound is MK-8776. In some embodiments, the compound is GDC-0425. In some embodiments, the compound is SAR 020106. In some embodiments, the compound is V-158411. In some embodiments, the compound is XL-844. In some embodiments, the compound is ARRY 575. In some embodiments, the compound is CASC-578. In some embodiments, the compound is LY-2880070. In some embodiments, the compound is CCT-245737. In some embodiments, the compound is prexasertib. In some embodiments, the compound is VER-250840. In some embodiments, the compound is BML-277. In some embodiments, the compound is GDC-0575. In some embodiments, the compound is CCT-241533.

In some embodiments, the compound is not PF-477736. In some embodiments, the compound is not rabusertib. In some embodiments, the compound is not AB-IsoG (isogranulatimide). In some embodiments, the compound is not AZD-7762. In some embodiments, the compound is not CCT-244747. In some embodiments, the compound is not CHK1-A. In some embodiments, the compound is not GNE-900. In some embodiments, the compound is not MK-8776. In some embodiments, the compound is not GDC-0425. In some embodiments, the compound is not SAR 020106. In some embodiments, the compound is not V-158411. In some embodiments, the compound is not XL-844. In some embodiments, the compound is not ARRY 575. In some embodiments, the compound is not CASC-578. In some embodiments, the compound is not LY-2880070. In some embodiments, the compound is not CCT-245737. In some embodiments, the compound is not prexasertib. In some embodiments, the compound is not VER-250840. In some embodiments, the compound is not BML-277. In some embodiments, the compound is not GDC-0575. In some embodiments, the compound is not CCT-241533.

### Therapeutically Effective Amount

The therapeutically effective amount of the agent to be administered in the method provided herein can depend on the agent (e.g., bioavailability, toxicity, ADME profile, efficacy, formulation, dosage form), subject (e.g., species, gender, body weight, age, diet), route and time of administration, severity of IPF, and result sought.

The therapeutically effective amount of an agent can be determined. Non-limiting examples of suitable methods include in vitro Chk1 binding assays (e.g., using fluorescence resonance energy transfer [FRET] or AlphaScreen amplified luminescent proximity homogeneous assay]), cell-free and cellular Chk1 kinase inhibition assays (e.g., to determine IC50 from amount of inhibition of phosphorylation of an exogenous substrate) and dosing in animal models (e.g., to determine MTD and ED50; using, for example, the TGF-β adenovirus transduction model, the radiation-induced fibrosis model, the bleomycin model [Hecker L et al., NADPH Oxidase-4 Mediates Myofibroblast Activation and Fibrogenic Responses to Lung Injury. Nat. Med., 15(9):1077-81, 2009]), and kinase selectivity profiling assays (e.g., to determine specificity). In addition to these assays, other assays can be utilized, and an assay can be modified for a particular application. Data obtained from cell culture assays and animal models can be used in formulating a range of dosages for testing in subjects (e.g., humans).

A therapeutically effective amount can lie within a range of circulating concentrations that include the ED50. HPLC assays or bioassays can be used to determine plasma concentrations.

In the treatment of an emergency, the agent can need to be administered at an amount that approaches the MTD to obtain a rapid response.

In some embodiments, a therapeutically-effective amount of a compound of the disclosure can reduce macrophage expression of an activation marker, reduce fibroblast-to-myofibroblast differentiation, reduce collagen deposition, reduce macrophage expression of a cytokine, reduce macrophage expression of a pro-fibrotic mediator, reduce epithelial cell expression of a senescence-associated gene, or a combination thereof, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.1%, 99.3%, 99.4%, 99.5%, 99.6%, 99.7%, 99.8%, 99.9%, 99.95%, 99.99%, or 100% relative to a control (e.g., a control not treated with the compound).

In some embodiments, a therapeutically-effective amount of a compound of the disclosure can reduce macrophage expression of an activation marker, reduce fibroblast-to-myofibroblast differentiation, reduce collagen deposition, reduce macrophage expression of a cytokine, reduce macrophage expression of a pro-fibrotic mediator, reduce epithelial cell expression of a senescence-associated gene, or a combination thereof, by at least about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 110-fold, about 120-fold, about 130-fold, about 140-fold, about 150-fold, about 160-fold, about 170-fold, about 180-fold, about 190-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, or about 2000-fold relative to a control (e.g., a control not treated with the compound).

### Administering the Agent

The agent to be administered in the method provided herein can be administered by any of a variety of suitable routes.

Non-limiting examples of such routes include orally, buccally, rectally, topically, transdermally, subcutaneously, intravenously (bolus or infusion), intraperitoneally, intramuscularly, sublingually, by inhalation, by insufflation, intranasally, transmucosally, intratracheally (including by pulmonary inhalation), intrathecally, intralymphatically, intralesionally, and epidurally.

In embodiments in which the administering is performed by inhalation, an inhalation device can be used. Non-liming examples of inhalation devices include nebulizer, metered-dose inhaler (MDI), dry powder inhaler (DPI), and dry powder nebulizer.

In some embodiments, the administering is done by controlled delivery (i.e., release in a site-directed and/or time-dependent manner).

Administering can be performed, for example, once as a single dose, or a plurality of times as a plurality of doses. In some embodiments, the administering can be performed over one or more extended periods of times (e.g., over a day, a week, a month, a year, or multiples thereof) either as a single dose or as a plurality of doses.

In some embodiments, the administering is performed daily for a period of at least one week. In some embodiments, the administering is performed weekly for a period of at least one month. In some embodiments, the administering is performed monthly for a period of at least 2 months. In some embodiments, the administering is performed daily, weekly, or monthly for a period of at least one year. In some embodiments, the administering is performed at least once monthly. In some such embodiments, the administering is performed between 1 and 2 times per month. In some embodiments, the administering is performed at least once weekly. In some such embodiments, the administering is performed between 1 and 4 times per week. In some embodiments, the administering is performed at least once daily. In some such embodiments, the administering is performed between 1 and 5 times per day.

Dosage amount and interval can be adjusted individually to provide plasma levels that are sufficient to maintain the minimal effective concentration (MEC). Compounds can be administered using a regimen that maintains plasma levels above the MEC for 5-100% of the time, e.g., between 20-90%, 30-90%, or 50-90% of the time until the desired amelioration of symptoms is achieved.

Pharmaceutical compositions described herein can be in unit dosage forms suitable for administration of precise dosages. In unit dosage form, the formulation can be divided into or dispensed as unit doses containing appropriate quantities of one or more compounds. The unit dosage can be in the form of a package containing discrete quantities of the formulation. Non-limiting examples are pills, capsules, tablets, and liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi dose containers with a preservative. Multiple unit doses can be dispensed, for example, from an inhaler.

A compound described herein can be present in a composition in a range of from about 1 μg to about 2000 mg; from about 100 μg to about 2000 mg; from about 100 μg to about 1000 mg; from about 100 μg to about 1 mg; from about 500 μg to about 1 mg; from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 μg, about 10 μg, about 100 μg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 1 mg/kg to about 1000 mg/kg, 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, about 150 mg/kg to about 200 mg/kg, or about 200 mg/kg to about 1000 mg/kg. In some embodiments, the amount of a compound that is administered to a subject can be about 0.01-10 mg/kg, about 0.01-20 mg/kg, about 0.01-50 mg/kg, about 0.1-10 mg/kg, about 0.1-20 mg/kg, about 0.1-50 mg/kg, about 0.1-100 mg/kg, about 0.5-10 mg/kg, about 0.5-20 mg/kg, about 0.5-50 mg/kg, about 0.5-100 mg/kg, about 1-10 mg/kg, about 1-20 mg/kg, about 1-50 mg/kg, or about 1-100 mg/kg body weight of the subject. In some embodiments, the amount of the compound administered is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg body weight of the subject.

In some embodiments, the amount of a compound that is administered to a subject can be about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg per body weight of the subject.

### Pharmaceutical Compositions

A pharmaceutical composition of the invention can be used, for example, before, during, or after treatment of a subject with another pharmaceutical agent.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition can improve the stability of a compound and can facilitate administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate tablets, pills, capsules, dragees, liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, and/or lubricants such as talc or magnesium stearate and, stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration. For buccal or sublingual administration, the compositions can be tablets, lozenges, or gels.

An enteric-coating can protect the contents of the oral formulation, for example, tablet, pill, or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, plant fibers, and Capsugel DR. The packaging technology in maintaining the potency may be Bel-Art, Biorx, ColorSafe, CSP Vials, Dynalon, MP Vials, PSA, Pill Pod, Qorpak, Safer Lock, or Wheaton. In some embodiments, the enteric coating is formed by a pH sensitive polymer. In some embodiments, the enteric coating is formed by eudragit FS30D. The enteric coated capsule may comprise at least about 1, 2, 3, 4, 5, 6, 7, 8, 9, or 10 enteric coatings. The enteric coating can be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include tablet, capsule, pill, liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after an antibiotic. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected.

A therapeutic agent can be administered for any length of time. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 20 years, or more. In some embodiments, a compound can be administered for the rest of a subject's life. In some embodiments, a compound can be administered for a length of time necessary to treat the disease (e.g., reduce symptoms or slow progression of the disease). The length of treatment can vary for each subject.

In some embodiments, the length of time a compound can be administered can be at least about 1 day, 2 days, 3 days, 4 days, 5 days, 6 days, 1 week, 2 weeks, 3 weeks, 4 weeks, 1 month, 5 weeks, 6 weeks, 7 weeks, 8 weeks, 2 months, 9 weeks, 10 weeks, 11 weeks, 12 weeks, 3 months, 13 weeks, 14 weeks, 15 weeks, 16 weeks, 4 months, 17 weeks, 18 weeks, 19 weeks, 20 weeks, 5 months, 21 weeks, 22 weeks, 23 weeks, 24 weeks, 6 months, 7 months, 8 months, 9 months, 10 months, 11 months, 1 year, 13 months, 14 months, 15 months, 16 months, 17, about 18 months, 19 months, 20 months, 21 months, 22 months 23 months, 2 years, 2.5 years, 3 years, 3.5 years, 4 years, 4.5 years, 5 years, 6 years, 7 years, 8 years, 9 years, 10 years, 11 years, 12, years, 13 years, 14 years, 15 years, 20 years, or more. In some embodiments, a compound can be administered for the rest of a subject's life.

In some embodiments, a pharmaceutically-acceptable amount of a compound of the disclosure is administered to a subject gradually over a period of time. In some embodiments, an amount of a compound of the disclosure can be administered to a subject gradually over a period of from about 0.1 h to about 24 h. In some embodiments, an amount of a compound of the disclosure can be administered to a subject over a period of about 0.1 h, about 0.2 h, about 0.3 h, about 0.4 h, about 0.5 h, about 0.6 h, about 0.7 h, about 0.8 h, about 0.9 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h. In some embodiments, a pharmaceutically-acceptable amount of a compound of the disclosure is administered gradually over a period of about 0.5 h. In some embodiments, a pharmaceutically-acceptable amount of a compound of the disclosure is administered gradually over a period of about 1 h. In some embodiments, a pharmaceutically-acceptable amount of a compound of the disclosure is administered gradually over a period of about 1.5 h.

Pharmaceutical compositions described herein can be administered 1, 2, 3, 4, 5, 6, 7, 8, 9, 10, 11, 12, 13, 14, 15, 16, 17, 18, 19, 20 or more times, and they can be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Pharmaceutical compositions described herein can be in unit dosage forms suitable for administration of precise dosages. In unit dosage form, the formulation can be divided into or dispensed as unit doses containing appropriate quantities of one or more compounds. The unit dosage can be in the form of a package containing discrete quantities of the formulation. Non-limiting examples are packaged injectables, vials, ampoules, pills, capsules, and tablets. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative. Multiple unit doses can be dispensed, for example, from an inhaler.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Combination Therapy

In some embodiments, the therapeutically effective amount of the agent that reduces or eliminates the kinase activity of Chk1 is administered together with a therapeutically effective amount of an additional agent. In some embodiments, the combination therapy can produce a significantly better therapeutic result than the additive effects achieved by each individual constituent when administered alone at a therapeutic dose.

Accordingly, in some embodiments, the present disclosure provides a method for treating idiopathic pulmonary fibrosis, the method comprising administering to a subject in need thereof (a) an effective amount of a Chk1 inhibitor of the disclosure and (b) an effective amount of at least one additional pharmaceutically active agent, for example, any additional therapeutic agent described herein, to provide a combination therapy.

The additional agents can be any therapeutic agent recognized as being useful for treating IPF or comorbidities thereof. Non-limiting examples of such therapeutic agents include immunomodulatory agents, cytokine suppressive anti-inflammatory drugs (CSAIDs; e.g., antibodies to or antagonists of human cytokines or growth factors [e.g., VEGF, FGF, PDGF], pirfenidone, nintedanib), inhibitors of other kinase activities (e.g., inhibitors of the kinase activity of Foxo1 and/or AurKA and/or AurKB and/or AurKC), and derivatives and prodrugs thereof.

The additional agent also can be an agent that imparts a beneficial attribute to the agent that reduces or eliminates the kinase activity of Chk1.

Such combination therapy can advantageously facilitate the use of a reduced dose of the agent that reduces or eliminates the kinase activity of Chk1 and/or of the additional agent.

In some embodiments, the dosage of the Chk1 inhibitor or additional therapeutic agent, for example, any additional therapeutic agent described herein, in combination therapy can be reduced as compared to monotherapy with each agent, while still achieving an overall therapeutic effect. In some embodiments, a Chk1 inhibitor and an additional therapeutic agent, for example, any additional therapeutic agent described herein, can exhibit a synergistic effect. In some embodiments, the synergistic effect of a Chk1 inhibitor and additional therapeutic agent, for example, any additional therapeutic agent described herein, can be used to reduce the total amount drugs administered to a subject, which decrease side effects experienced by the subject.

A Chk1 inhibitor of the disclosure can be used in combination with at least one additional pharmaceutically-active agent, for example, any additional therapeutic agent described herein. In some embodiments, the at least one additional pharmaceutically-active agent, for example, any additional therapeutic agent described herein, can modulate the same or a different target as the Chk1 inhibitor. In some embodiments, the at least one additional pharmaceutically-active agent, for example, any additional therapeutic agent described herein, can modulate the same target as the Chk1 inhibitor of the disclosure, or other components of the same pathway, or overlapping sets of target enzymes. In some embodiments, the at least one additional pharmaceutically-active agent, for example, any additional therapeutic agent described herein, can modulate a different target from the Chk1 inhibitor of the disclosure

The additional agent can be administered at different times during the course of therapy (i.e., before or after administering the agent that reduces or eliminates the kinase activity of Chk1), or it can be administered concurrently with the agent that reduces or eliminates the kinase activity of Chk1.

In some embodiments, the disclosure provides a method of treating idiopathic pulmonary fibrosis (IPF) comprising the step of administering to a subject needing treatment for IPF a therapeutically effective amount of an agent that reduces or eliminates the kinase activity of checkpoint kinase 1 (Chk1). In some embodiments, the method is effective at reducing at least one pathology in the pulmonary tissue of the subject selected from the group consisting of: an aberrant rate of proliferation of fibroblasts; an aberrant rate of differentiation of fibroblasts into myofibroblasts; an aberrant formation of fibrotic loci; an aberrant deposition of an ECM protein; an aberrant rate of myofibroblast contraction; an aberrant rate of myofibroblast apoptosis; an aberrant attachment of myofibroblasts to an ECM; an aberrant production of a cytokine; an aberrant inflammation; an aberrant growth of scar tissue; and an aberrant expression of a senescence-associated gene.

### Embodiments

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of an activation marker in the subject by at least about 5% relative to a control. In some embodiments, the control is macrophage expression of the activation marker in a control subject that was not administered the Chk1 inhibitor. In some embodiments, the control is macrophage expression of the activation marker in the subject prior to the administering the Chk1 inhibitor. In some embodiments, the activation marker is an M1 macrophage activation marker. In some embodiments, the activation marker is an M2 macrophage activation marker. In some embodiments, the activation marker is CD80. In some embodiments, the activation marker is CD163. In some embodiments, the macrophage expression of the activation marker is as determined by contacting macrophages with LPS and interferon gamma, staining the macrophages with a fluorescently-conjugated antibody specific for CD80, and determining mean fluorescence intensity of the macrophages for the CD80 via flow cytometry. In some embodiments, the macrophage expression of the activation marker is as determined by contacting macrophages with IL-4, staining the macrophages with a fluorescently-conjugated antibody specific for CD163, and determining mean fluorescence intensity of the macrophages for the CD163 via flow cytometry. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces a level of fibroblast to myofibroblast differentiation in the subject by at least about 5% relative to a control. In some embodiments, the control is a level of fibroblast to myofibroblast differentiation in a control subject that was not administered the Chk1 inhibitor. In some embodiments, the control is a level of fibroblast to myofibroblast differentiation in the subject prior to the administering the Chk1 inhibitor. In some embodiments, the level of fibroblast to myofibroblast differentiation is as determined by quantifying expression of alpha smooth muscle actin after treating fibroblasts with TGF-β. In some embodiments, the level of fibroblast to myofibroblast differentiation is as determined by contacting fibroblasts with TGF-β, staining the fibroblasts with a reagent that specifically stains alpha smooth muscle actin, and conducting high content analysis to determine percent inhibition of alpha smooth muscle actin induction. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces collagen deposition in a lung of the subject by at least about 5% relative to a control subject that was not administered the Chk1 inhibitor. In some embodiments, the Chk1 inhibitor reduces a collagen fiber count in the lung of the subject by at least about 5% relative to the control subject. In some embodiments, the Chk1 inhibitor reduces a collagen fiber density in the lung of the subject by at least about 5% relative to the control subject. In some embodiments, the Chk1 inhibitor reduces a level of collagen fiber alignment in the lung of the subject by at least about 5% relative to the control subject. In some embodiments, the Chk1 inhibitor reduces an amount of collagen in the lung of the subject by at least about 5% relative to the control subject. In some embodiments, the collagen deposition is as quantified by imaging Sirius red stained histological sections from a lung biopsy and determining a surface area that stains positive for Sirius red. In some embodiments, the collagen deposition is as quantified by imaging Sirius red stained histological sections from a lung biopsy and determining a collagen fiber count. In some embodiments, the collagen deposition is as quantified by imaging Sirius red stained histological sections from a lung biopsy and determining a collagen fiber density. In some embodiments, the collagen deposition is as quantified by imaging Sirius red stained histological sections from a lung biopsy and determining a level of collagen fiber alignment. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of a cytokine in a lung of the subject by at least about 5% relative to a control. In some embodiments, the control is macrophage expression of the cytokine in a lung of a control subject that was not administered the Chk1 inhibitor. In some embodiments, the control is macrophage expression of the cytokine in the lung of the subject prior to the administering the Chk1 inhibitor. In some embodiments, the cytokine is a pro-inflammatory cytokine. In some embodiments, the cytokine is an anti-inflammatory cytokine. In some embodiments, the cytokine is a chemotactic cytokine. In some embodiments, the cytokine is IL-6. In some embodiments, the cytokine is IL-10. In some embodiments, the cytokine is IL-12p40. In some embodiments, the cytokine is TNF-α. In some embodiments, the cytokine is RANTES. In some embodiments, the macrophage expression of the cytokine is as determined by contacting macrophages with LPS and interferon gamma and determining an amount of the cytokine produced via multiplex immunoassay. In some embodiments, the macrophage expression of the cytokine is as determined by contacting macrophages with IL-4 and determining an amount of the cytokine produced via multiplex immunoassay. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of a pro-fibrotic mediator in a lung of the subject by at least about 5% relative to a control. In some embodiments, the control is macrophage expression of the pro-fibrotic mediator in a lung of a control subject that was not administered the Chk1 inhibitor. In some embodiments, the control is macrophage expression of the pro-fibrotic mediator in the lung of the subject prior to the administering the Chk1 inhibitor. In some embodiments, the pro-fibrotic mediator is a matrix metalloproteinase. In some embodiments, the pro-fibrotic mediator is MMP2. In some embodiments, the macrophage expression of the pro-fibrotic mediator is as determined by contacting macrophages with LPS and interferon gamma and determining an amount of the pro-fibrotic mediator produced via multiplex immunoassay. In some embodiments, the macrophage expression of the pro-fibrotic mediator is as determined by contacting macrophages with IL-4 and determining an amount of the pro-fibrotic mediator produced via multiplex immunoassay. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces epithelial cell expression of a senescence-associated gene in a lung of the subject by at least about 5% relative to a control. In some embodiments, the control is epithelial cell expression of the senescence-associated gene in a lung of a control subject that was not administered the Chk1 inhibitor. In some embodiments, the control is epithelial cell expression of the senescence-associated gene in the lung of the subject prior to the administering the Chk1 inhibitor. In some embodiments, the senescence associated gene encodes a senescence-associated secreted protein. In some embodiments, the epithelial cell is an alveolar type 1 epithelial cell. In some embodiments, the epithelial cell is an alveolar type 2 epithelial cell. In some embodiments, the epithelial cell is a basal epithelial cell. In some embodiments, the epithelial cell is a ciliated epithelial cell. In some embodiments, the epithelial cell is a club epithelial cell. In some embodiments, the epithelial cell is a goblet cell. In some embodiments, the epithelial cell expression of the senescence-associated gene is as determined by contacting epithelial cells with bleomycin and determining an amount of the senescence-associated gene expressed via RNA sequencing. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces macrophage expression of an activation marker in the subject by at least about 5%, reduces a level of fibroblast to myofibroblast differentiation in the subject by at least about 5%, reduces collagen deposition in a lung of the subject by at least about 5%, reduces macrophage expression of a cytokine in the lung of the subject by at least about 5%, reduces macrophage expression of a pro-fibrotic mediator in the lung of the subject by at least about 5%, and reduces epithelial cell expression of a senescence-associated gene in the lung of the subject by at least about 5% relative to a control, wherein the control is a control subject that was not administered the Chk1 inhibitor. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 μg/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of treating idiopathic pulmonary fibrosis (IPF) in a subject in need thereof, comprising administering to the subject a therapeutically-effective amount of a pharmaceutical composition, wherein the pharmaceutical composition comprises a Chk1 inhibitor, wherein the Chk1 inhibitor is not a thiazole compound, a heterocyclic urea compound, a heterocyclic thiourea compound, or an anilinopiperazine compound. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the subject is a mammal. In some embodiments, the subject is a human. In some embodiments, the pharmaceutical composition further comprises a pharmaceutically-acceptable excipient. In some embodiments, the pharmaceutical composition is administered in a unit dosage form. In some embodiments, the method further comprises administering an additional therapeutic agent to the subject. In some embodiments, the pharmaceutical composition further comprises an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM. In some embodiments, the therapeutically-effective amount is from about 1 μg to about 1 gram. In some embodiments, the therapeutically-effective amount is from about 0.1 m/kg to about 100 mg/kg. In some embodiments, the pharmaceutical composition is administered orally. In some embodiments, the pharmaceutical composition is administered intravenously. In some embodiments, the pharmaceutical composition is administered via inhalation. In some embodiments, the pharmaceutical composition is administered intranasally. In some embodiments, the pharmaceutical composition is administered topically. In some embodiments, the pharmaceutical composition is administered subcutaneously. In some embodiments, the pharmaceutical composition is administered transmucosally. In some embodiments, the pharmaceutical composition is administered intraperitoneally. In some embodiments, the pharmaceutical composition is administered intramuscularly. In some embodiments, the Chk1 inhibitor in the pharmaceutical composition is in a prodrug form.

Disclosed herein, in some embodiments, is a method of reducing activation of a macrophage, comprising contacting a population of cells with a Chk1 inhibitor, wherein the population of cells comprises the macrophage, wherein upon contacting the population of cells with the Chk1 inhibitor, the macrophage exhibits an expression level of an activation marker that is at least about 5% lower than an expression level of the activation marker by a macrophage that was not contacted with the Chk1 inhibitor. In some embodiments, the activation marker is an M1 macrophage activation marker. In some embodiments, the activation marker is an M2 macrophage activation marker. In some embodiments, the activation marker is CD80. In some embodiments, the activation marker is CD163. In some embodiments, the macrophage is from a lung. In some embodiments, the macrophage is from a lung of a subject with idiopathic pulmonary fibrosis. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the population of cells with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

Disclosed herein, in some embodiments, is a method of reducing differentiation of a fibroblast into a myofibroblast, comprising contacting a population of cells with a Chk1 inhibitor, wherein the population of cells comprises the fibroblast, wherein upon contacting the population of cells with the Chk1 inhibitor, the fibroblast exhibits an expression level of alpha smooth muscle actin that is at least about 5% lower than an expression level of the alpha smooth muscle actin by a fibroblast that was not contacted with the Chk1 inhibitor. In some embodiments, the differentiation of the fibroblast into the myofibroblast is as determined by quantifying expression of alpha smooth muscle actin after contacting the fibroblast with TGF-β. In some embodiments, the fibroblast is from a lung. In some embodiments, the fibroblast is from a lung of a subject with idiopathic pulmonary fibrosis. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the population of cells with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

Disclosed herein, in some embodiments, is a method of reducing collagen deposition, comprising contacting a tissue with a Chk1 inhibitor, wherein upon contacting the tissue with the Chk1 inhibitor, the tissue exhibits an at least about 5% lower level of an indicator of collagen deposition relative to a tissue that was not contacted with the Chk1 inhibitor. In some embodiments, the indicator of collagen deposition is a collagen fiber count. In some embodiments, the indicator of collagen deposition is a collagen fiber density. In some embodiments, the indicator of collagen deposition is a level of collagen fiber alignment. In some embodiments, the indicator of collagen deposition is an amount of collagen. In some embodiments, the indicator of collagen deposition is as quantified by imaging Sirius red stained histological sections of the tissue and determining a surface area that stains positive for Sirius red. In some embodiments, the indicator of collagen deposition is as quantified by imaging Sirius red stained histological sections of the tissue and determining a collagen fiber count. In some embodiments, the indicator of collagen deposition is as quantified by imaging Sirius red stained histological sections of the tissue and determining a collagen fiber density. In some embodiments, the indicator of collagen deposition is as quantified by imaging Sirius red stained histological sections of the tissue and determining a level of collagen fiber alignment. In some embodiments, the tissue is a lung. In some embodiments, the tissue is a lung of a subject with idiopathic pulmonary fibrosis. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the tissue with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

Disclosed herein, in some embodiments, is a method of reducing a level of a cytokine, comprising contacting a population of cells with a Chk1 inhibitor, wherein the population of cells comprises a macrophage, wherein upon contacting the population of cells with the Chk1 inhibitor, the macrophage produces a level of the cytokine that is at least about 5% lower than a level of the cytokine produced by a macrophage that was not contacted with the Chk1 inhibitor. In some embodiments, the cytokine is a pro-inflammatory cytokine. In some embodiments, the cytokine is an anti-inflammatory cytokine. In some embodiments, the cytokine is a chemotactic cytokine. In some embodiments, the cytokine is IL-6. In some embodiments, the cytokine is IL-10. In some embodiments, the cytokine is IL-12p40. In some embodiments, the cytokine is TNF-α. In some embodiments, the cytokine is RANTES. In some embodiments, the macrophage is from a lung. In some embodiments, the macrophage is from a lung of a subject with idiopathic pulmonary fibrosis. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the population of cells with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

Disclosed herein, in some embodiments, is a method of reducing a level of a pro-fibrotic mediator, comprising contacting a population of cells with a Chk1 inhibitor, wherein the population of cells comprises a macrophage, wherein upon contacting the population of cells with the Chk1 inhibitor, the macrophage produces a level of the pro-fibrotic mediator that is at least about 5% lower than a level of the pro-fibrotic mediator produced by a macrophage that was not contacted with the Chk1 inhibitor. In some embodiments, the pro-fibrotic mediator is a matrix metalloproteinase. In some embodiments, the pro-fibrotic mediator is MMP2. In some embodiments, the macrophage is from a lung. In some embodiments, the macrophage is from a lung of a subject with idiopathic pulmonary fibrosis. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the population of cells with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

Disclosed herein, in some embodiments, is a method of reducing cellular senescence, comprising contacting a population of cells with a Chk1 inhibitor, wherein the population of cells comprises an epithelial cell, wherein upon contacting the population of cells with the Chk1 inhibitor, the epithelial cell expresses a level of a senescence-associated gene that is at least about 5% lower than a level of the senescence-associated gene expressed by an epithelial cell that was not contacted with the Chk1 inhibitor. In some embodiments, the senescence associated gene encodes a senescence-associated secreted protein. In some embodiments, the epithelial cell is from a subject that has idiopathic pulmonary fibrosis. In some embodiments, the epithelial cell is an epithelial cell type that is enriched in a lung of a subject with idiopathic pulmonary fibrosis. In some embodiments, the epithelial cell is an alveolar type 1 epithelial cell. In some embodiments, the epithelial cell an alveolar type 2 epithelial cell. In some embodiments, the epithelial cell is a basal epithelial cell. In some embodiments, the epithelial cell is a ciliated epithelial cell. In some embodiments, the epithelial cell is a club epithelial cell. In some embodiments, the epithelial cell is a goblet cell. In some embodiments, the contacting occurs inside a human subject. In some embodiments, the Chk1 inhibitor is not a thiazole compound, a heterocyclic urea compound, a heterocyclic thiourea compound, or an anilinopiperazine compound. In some embodiments, the Chk1 inhibitor is CCT-245737. In some embodiments, the Chk1 inhibitor is VER-250840. In some embodiments, the Chk1 inhibitor is AB-IsoG (isogranulatimide). In some embodiments, the Chk1 inhibitor is AZD-7762. In some embodiments, the Chk1 inhibitor is CCT-244747. In some embodiments, the Chk1 inhibitor is CHK1-A. In some embodiments, the Chk1 inhibitor is GNE-900. In some embodiments, the Chk1 inhibitor is MK 8776. In some embodiments, the Chk1 inhibitor is PF-477736. In some embodiments, the Chk1 inhibitor is rabusertib. In some embodiments, the Chk1 inhibitor is GDC-0425. In some embodiments, the Chk1 inhibitor is SAR 020106. In some embodiments, the Chk1 inhibitor is V-158411. In some embodiments, the Chk1 inhibitor is XL-844. In some embodiments, the Chk1 inhibitor is ARRY 575. In some embodiments, the Chk1 inhibitor is CASC-578. In some embodiments, the Chk1 inhibitor is LY-2880070. In some embodiments, the Chk1 inhibitor is prexasertib. In some embodiments, the Chk1 inhibitor is GDC-0575. In some embodiments, the Chk1 inhibitor is BML-277. In some embodiments, the Chk1 inhibitor is CCT-241533. In some embodiments, the method further comprises contacting the population of cells with an additional therapeutic agent. In some embodiments, the additional therapeutic agent comprises nintedanib. In some embodiments, the additional therapeutic agent comprises pirfenidone. In some embodiments, the additional therapeutic agent comprises an immunomodulatory agent. In some embodiments, the Chk1 inhibitor inhibits Chk1 with an IC50 of less than about 10 μM.

It is to be understood that, while the invention has been described in conjunction with certain specific embodiments thereof, the foregoing description is intended to illustrate and not limit the scope of the invention. Other aspects, advantages, and modifications within the scope of the invention will be apparent to those skilled in the art to which the invention pertains.

### EXAMPLES

The following examples are included to illustrate specific embodiments of the invention. The techniques disclosed in the examples represent techniques discovered by the inventors to function well in the practice of the invention; however, those of skill in the art should, in light of the present disclosure, appreciate that many changes can be made in the specific embodiments that are disclosed and still obtain a like or similar result without departing from the spirit and scope of the invention. Therefore, all matter set forth or shown in the examples is to be interpreted as illustrative and not in a limiting sense.

### Example 1: Transcriptome Analyses of IPF Fibroblasts/Myofibroblasts and Macrophages

Gene expression datasets from Gene Expression Omnibus (GEO; Barrett T et al. NCBI GEO: archive for functional genomics data sets—update; Nucleic Acids Res. 2013; 41(D1):D991-5) related to IPF were identified and processed to form characteristic direction (CD) vectors (Clark N R et al. The characteristic direction: a geometrical approach to identify differentially expressed genes. BMC Bioinformatics. 2014; 15:79) describing the gene expression perturbation imparted by the disease. From these datasets (GSE71351, GSE44723, GSE21369, GSE24206, GSE2052, GSE49072), expression vectors were identified related to a general IPF signature, a rapid versus slow progression phenotype in fibroblasts, and an early IPF signature.

In parallel, genetic perturbation data from the LINCS project (Subramanian A et al. A next generation connectivity map: L1000 platform and the first 1,000,000 profiles. Cell. 2017; 171(6):1437-1452) were processed using published methods (Lamb J et al. The Connectivity Map: using gene-expression signatures to connect small molecules, genes, and disease. Science. 2006; 313(5795):1929-35; Niepel M et al. Common and cell-type specific responses to anti-cancer drugs revealed by high throughput transcript profiling. Nat Commun. 2017; 8(1):1186) to create CD vectors for each genetic perturbation.

Using approaches described in Niepel et al., the cosine distance between each CD vector of the disease and each CD vector of the drug was calculated to obtain a measure of angle between the vectors (value of 1 when the vectors pointed in the same direction, value of −1 when they pointed in opposite directions). Since there are multiple genetic perturbations per gene, genes that most consistently imparted a perturbation that was opposite in direction to the disease in at least one condition (cell line, perturbagen concentration, exposure time) were identified. Chk1 had the desired behavior across all 3 IPF disease signatures.

These analyses identified Chk1 and various known inhibitors of Chk1 as potential therapeutic avenues for treatment of IPF. Similar analyses performed on other fibrotic conditions (e.g., Scleroderma, COPD, keloids, myelofibrosis, ulcerative colitis, uterine fibroids, cardiac fibrosis) did not identify Chk1 and its inhibitors.

### Example 2: Chk1 Inhibition Blocks Differentiation of Lung Fibroblasts Isolated from Human IPF Donor Lungs into Myofibroblasts

This example demonstrates that Chk1 inhibition blocks differentiation of lung fibroblasts isolated from human IPF donor lungs into myofibroblasts.

Primary fibroblasts cultured from the lungs of patients with IPF (n=3) or healthy donors (n=3) were exposed to Chk1 inhibitors or control compounds to determine the effect of Chk1 inhibitors on fibroblast differentiation into myofibroblasts (i.e., fibroblast-to-myofibroblast transition [FMT]). Cells were seeded in 96-well plates and were treated with 1.25 ng/mL TGFb to induce differentiation to a myofibroblast phenotype, characterized by the induction of alpha smooth muscle actin (aSMA). Chk1 inhibitors were dosed in an 8-point concentration curve 1 hr prior to TGFb treatment. Alpha-SMA and DAPI staining were assessed after 72 hours using high content analysis. The ability of the Chk1 inhibitors to block fibroblast-to-myofibroblast differentiation was determined by quantifying percent inhibition (PIN) of aSMA induction. Cell viability was also measured to determine whether the compounds were cytotoxic.

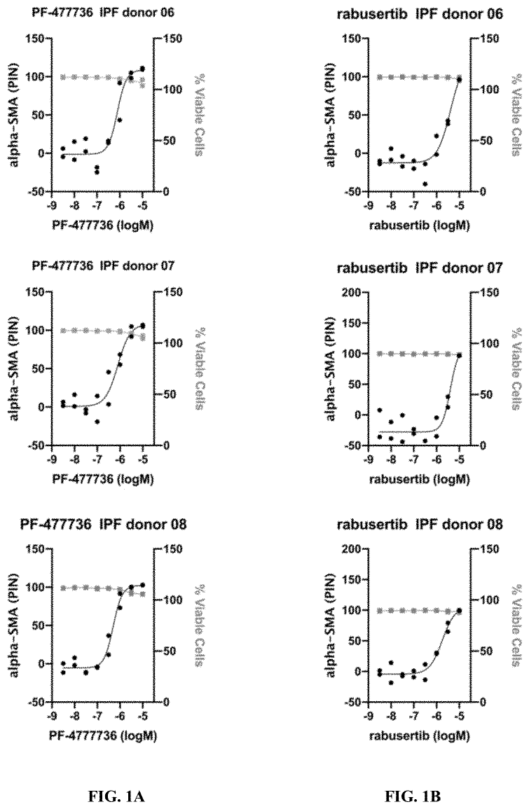

As shown in FIGS. 1A-B and 9-15, Chk1 inhibition blocked fibroblast differentiation into myofibroblasts in a dose-dependent manner, and independent of any impact on cell viability.

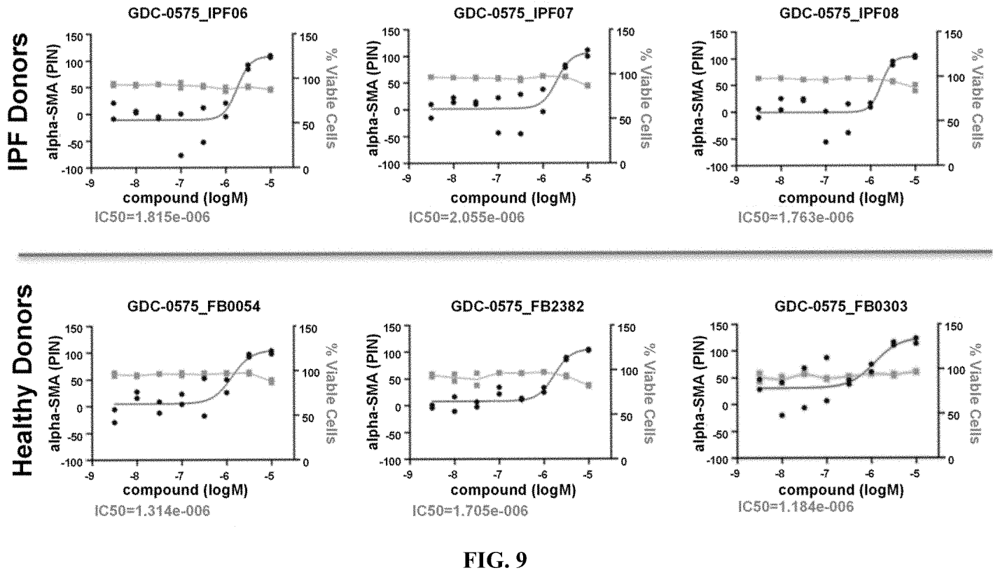

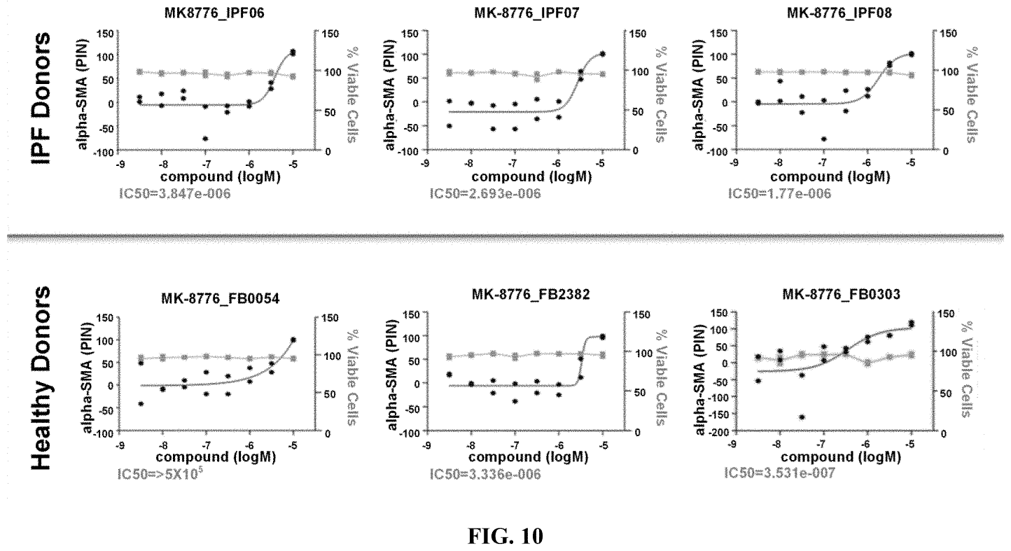

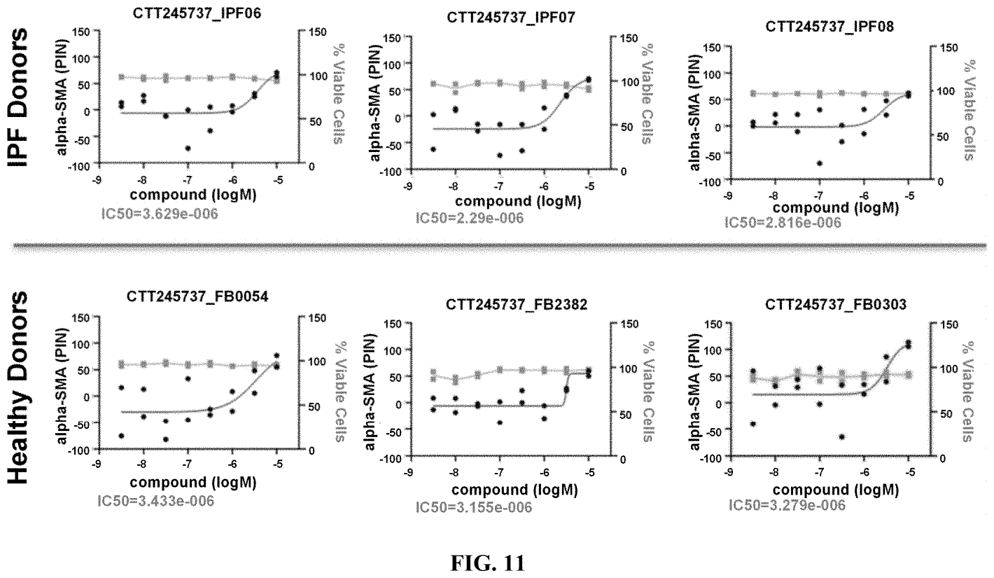

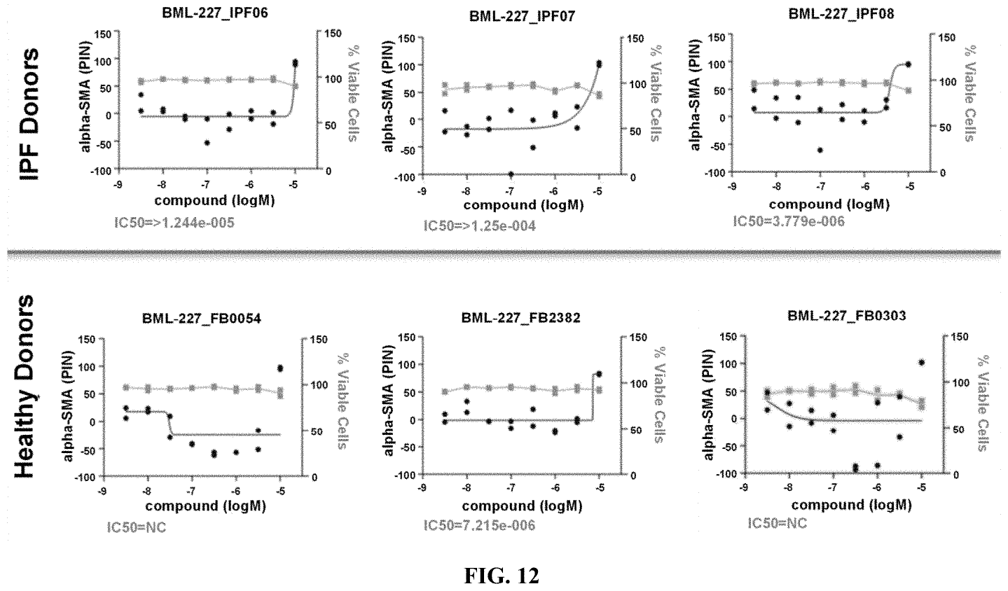

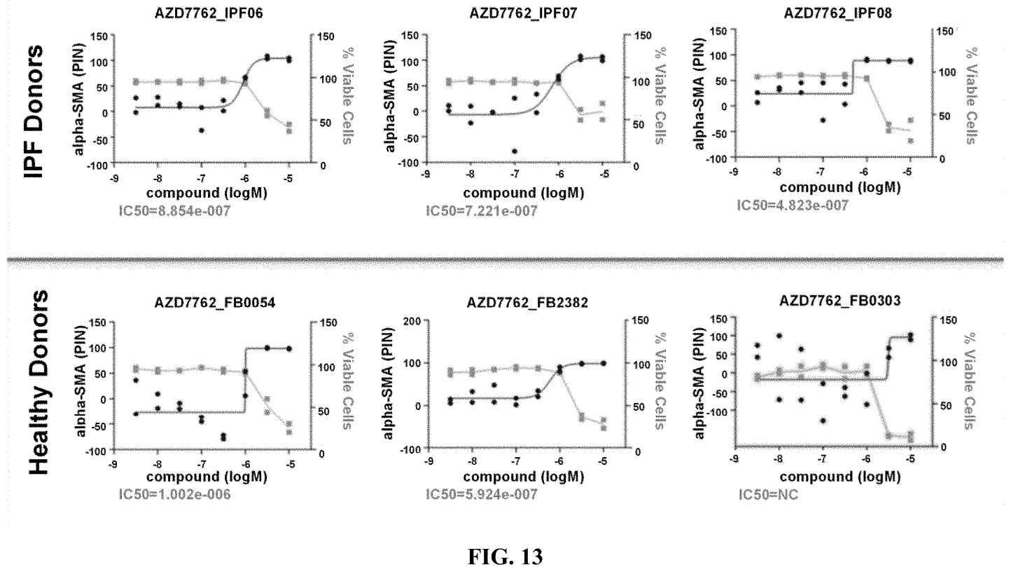

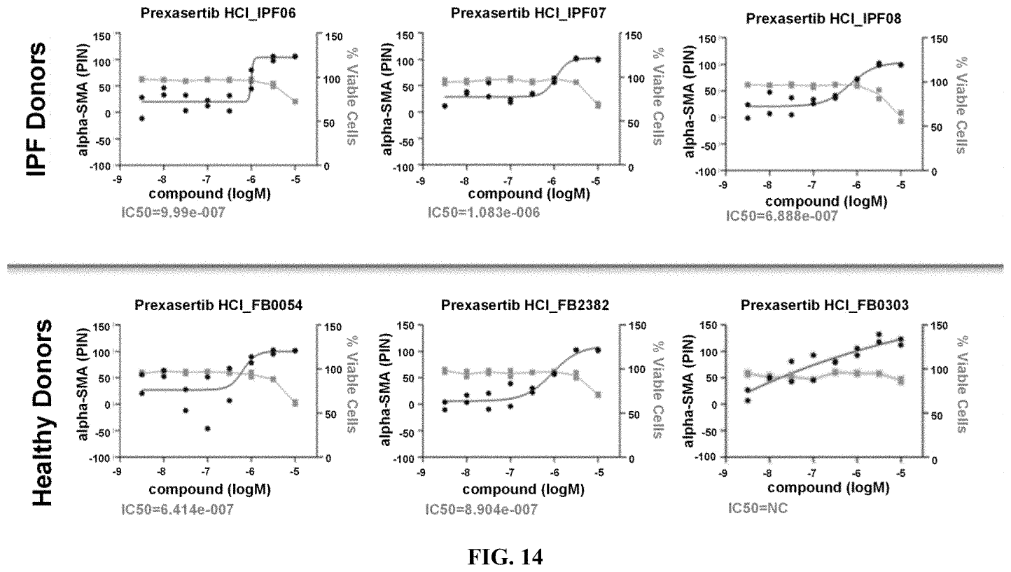

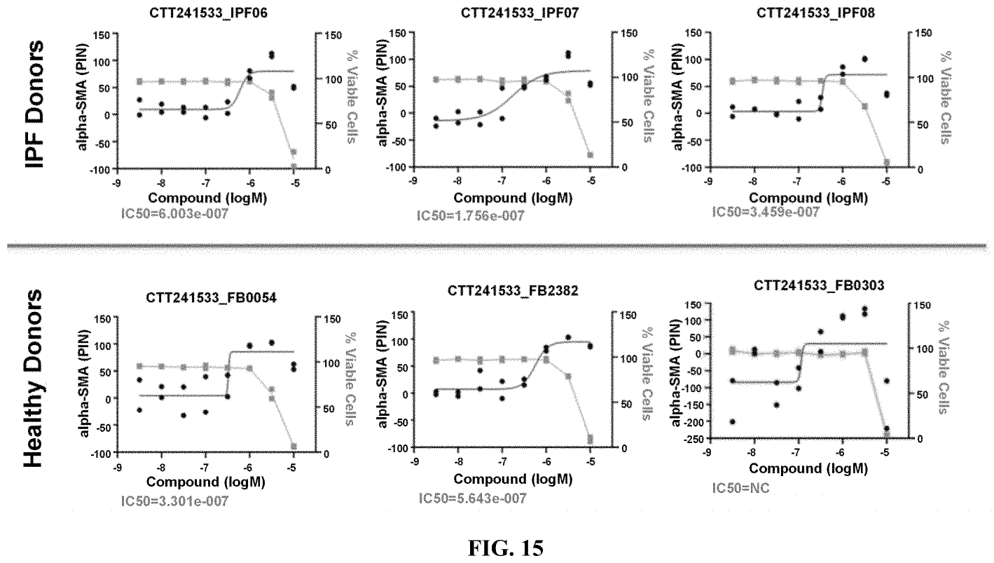

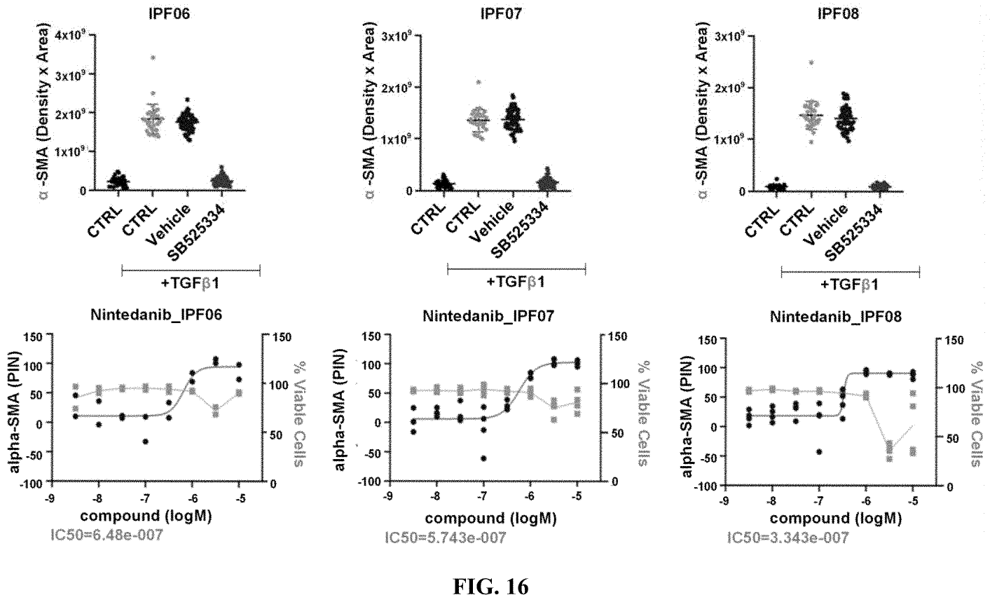

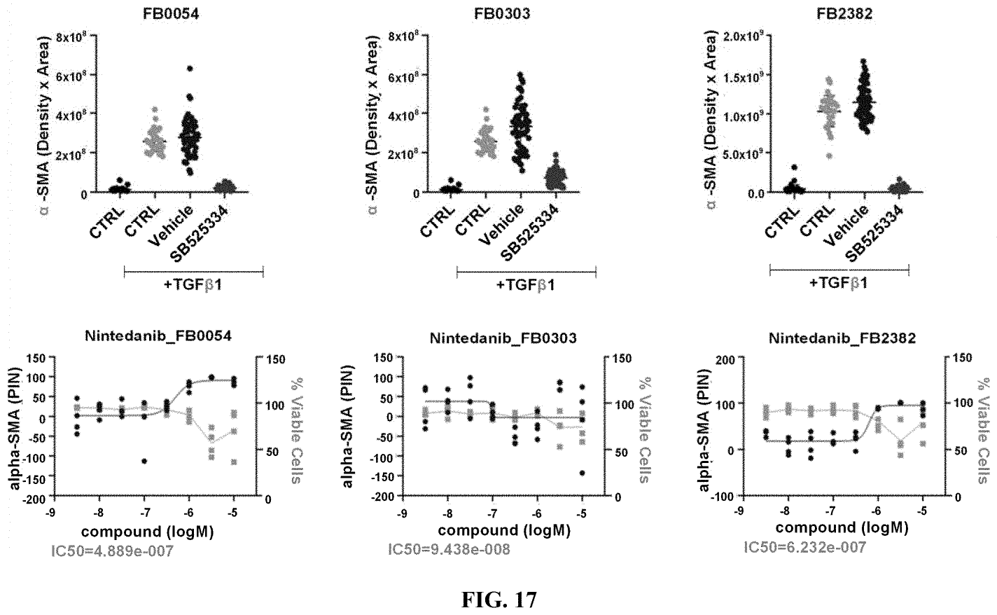

FIG. 1A illustrates the effects of PF-477736 on fibroblast-to-myofibroblast differentiation. FIG. 1B illustrates the effects of rabusertib on fibroblast-to-myofibroblast differentiation. FIG. 9 illustrates the effects of GDC-0575 on fibroblast-to-myofibroblast differentiation. FIG. 10 illustrates the effects of MK-8776 on fibroblast-to-myofibroblast differentiation. FIG. 11 illustrates the effects of CCT-245737 on fibroblast-to-myofibroblast differentiation. FIG. 12 illustrates the effects of BML-277 on fibroblast-to-myofibroblast differentiation. FIG. 13 illustrates the effects of AZD-7762 on fibroblast-to-myofibroblast differentiation. FIG. 14 illustrates the effects of prexasertib on fibroblast-to-myofibroblast differentiation. FIG. 15 illustrates the effects of CCT-241533 on fibroblast-to-myofibroblast differentiation. FIG. 16 provides control analysis of IPF donor samples dosed with control compounds. FIG. 17 provides control analysis of healthy controls dosed with control compounds. Subject FB0303 exhibited an aberrant response to nintedanib which could warrant exclusion of this subject from further analysis.

These methods can similarly be applied to test the ability of other Chk1 inhibitors to block fibroblast-to-myofibroblast differentiation.

Tables 1-7 provide IC50 data for inhibition of fibroblast to myofibroblast differentiation for certain Chk1 inhibitors.

TABLE 1
Table 1: IC50 data for GDC-0575 inhibition of
fibroblast to myofibroblast differentiation.

| Donor | Disease State | LogM IC50 aSMA |
|---|---|---|
| IPF06 | IPF | −5.7 |
| IPF07 | IPF | −5.7 |
| IPF08 | IPF | −5.8 |
| FB0054 | Healthy | −5.9 |
| FB2382 | Healthy | −5.9 |
| FB0303* | Healthy | −5.8 |

TABLE 2
Table 2: IC50 data for MK-8776 inhibition of
fibroblast to myofibroblast differentiation.

| Donor | Disease State | LogM IC50 aSMA |
|---|---|---|
| IPF06 | IPF | −5.7 |
| IPF07 | IPF | −5.7 |
| IPF08 | IPF | −5.8 |
| FB0054 | Healthy | >5 |
| FB2382 | Healthy | −5.5 |
| FB0303* | Healthy | −6.4 |

TABLE 3
Table 3: IC50 data for CCT-245737 inhibition of
fibroblast to myofibroblast differentiation.

| Donor | Disease State | LogM IC50 aSMA |
|---|---|---|
| IPF06 | IPF | −5.5 |
| IPF07 | IPF | −5.5 |
| IPF08 | IPF | −5.5 |
| FB0054 | Healthy | −5.5 |
| FB2382 | Healthy | −5.5 |
| FB0303* | Healthy | −5.6 |

TABLE 4
Table 4: IC50 data for BML-277 inhibition of
fibroblast to myofibroblast differentiation.

| Donor | Disease State | LogM IC50 aSMA |
|---|---|---|
| IPF06 | IPF | >−5 |
| IPF07 | IPF | >−5 |
| IPF08 | IPF | −5.4 |
| FB0054 | Healthy | NC |
| FB2382 | Healthy | −5.1 |
| FB0303* | Healthy | NC |

TABLE 5
Table 5: IC50 data for AZD-7762 inhibition of
fibroblast to myofibroblast differentiation.

| Donor | Disease State | LogM IC50 aSMA |
|---|---|---|
| IPF06 | IPF | −6.1 |
| IPF07 | IPF | −6.1 |
| IPF08 | IPF | −6.3 |
| FB0054 | Healthy | −5.9 |
| FB2382 | Healthy | −6.2 |
| FB0303* | Healthy | NC |

TABLE 6
Table 6: IC50 data for prexasertib inhibition
of fibroblast to myofibroblast differentiation.

| Donor | Disease State | LogM IC50 aSMA |
|---|---|---|
| IPF06 | IPF | −6 |
| IPF07 | IPF | −6 |
| IPF08 | IPF | −6.2 |
| FB0054 | Healthy | −6.2 |
| FB2382 | Healthy | −6.1 |
| FB0303* | Healthy | NC |

TABLE 7
Table 7: IC50 data for CCT-241533 inhibition of
fibroblast to myofibroblast differentiation.

| Donor | Disease State | LogM IC50 aSMA |
|---|---|---|
| IPF06 | IPF | −6.2 |
| IPF07 | IPF | −6.8 |
| IPF08 | IPF | −6.5 |
| FB0054 | Healthy | −6.4 |
| FB2382 | Healthy | −6.3 |
| FB0303* | Healthy | NC |

As the differentiation of fibroblasts into myofibroblasts is implicated in IPF pathogenesis, these data suggest that Chk1 inhibitors can be used to treat IPF.

### Example 3: Inhibition of Differentiation of Human Monocytes into Macrophages of M1 or M2 Phenotype Via Inhibition of Chk1

Human monocytes were isolated from the peripheral blood of IPF patients, exposed to various doses of PF-477736 for 1 hour, and then differentiated into M1 macrophages by exposure to LPS/IFNg, or into M2 macrophages by exposure to IL-4, for 3 days. The cells were analyzed by flow cytometry for CD80 cell surface expression as marker of M1 differentiation, and for CD163 cell surface expression as marker of M2 differentiation. In addition, cell supernatants were tested in a Luminex-based assay for various cytokines and matrix metalloproteinases (MMPs).

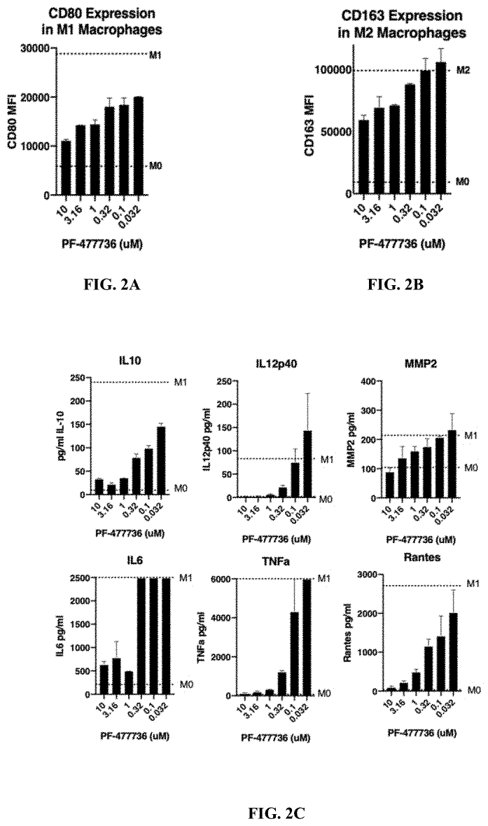

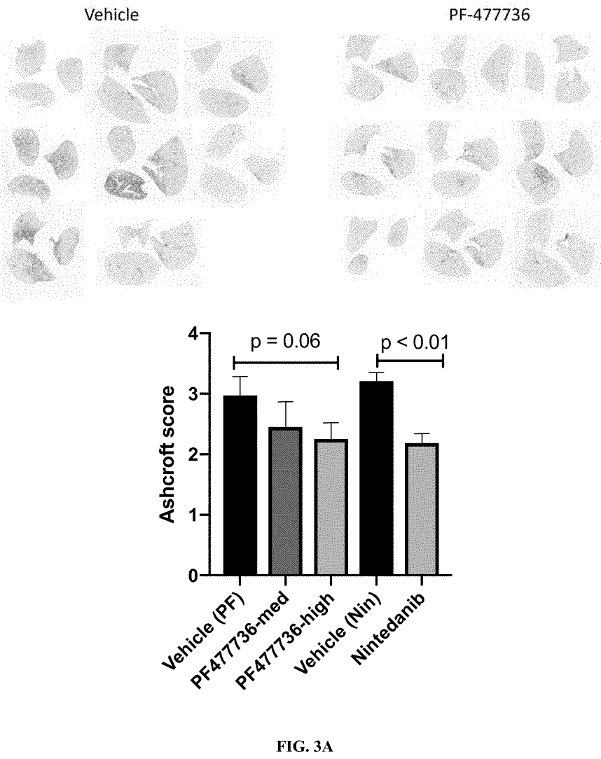

As shown in FIGS. 2A-C, Chk1 inhibition reduced the activation of macrophages towards either an M1 or M2 phenotype, and inhibited the production of a number of pro-fibrotic and pro-inflammatory mediators, including cytokines. FIG. 2A illustrates that PF-477736 inhibits M1 macrophage activation in a dose-dependent manner. FIG. 2B illustrates that PF-477736 inhibits M2 macrophage activation in a dose-dependent manner. FIG. 2C shows PF-477736 treatment results in lower levels of representative cytokines and matrix metalloproteinases in the supernatants of macrophages exposed to LPS and IFNg.

As M1 and M2 macrophage activation has been implicated in IPF pathogenesis, these data suggest that Chk1 inhibitors can be used to treat IPF.

These methods can similarly be applied to test the ability of other Chk1 inhibitors to inhibit macrophage activation, macrophage differentiation into M1 and/or M2 phenotypes, production of cytokines by macrophages, or production of pro-fibrotic mediators by macrophages. In some embodiments, the production of cytokines and/or pro-fibrotic mediators by M2 macrophages can be modulated (e.g., increased or decreased) by Chk1 inhibition.

### Example 4: Chk1 Inhibition Decreases Histopathology and Collagen Deposition in a Mouse Model of Pulmonary Fibrosis

To induce fibrosis, 6-8 week old C57BL6 mice were dosed with 3 mg/kg bleomycin hydrochloride in a volume of 50 μL per animal via intratracheal (i.t.) administration with a microsprayer. One week following administration of bleomycin, mice were treated with compound or vehicle once daily from day 7 to day 20. PF-477736 was administered at a dose of either 10 mg/kg (“med”) or 20 mg/kg (“high”) i.p. in a vehicle of 50 nM sodium acetate buffer and 4% dextrose, pH 4. Nintedanib was administered orally at 100 mg/kg in a vehicle of 1% methylcellulose. n=10 mice per group.

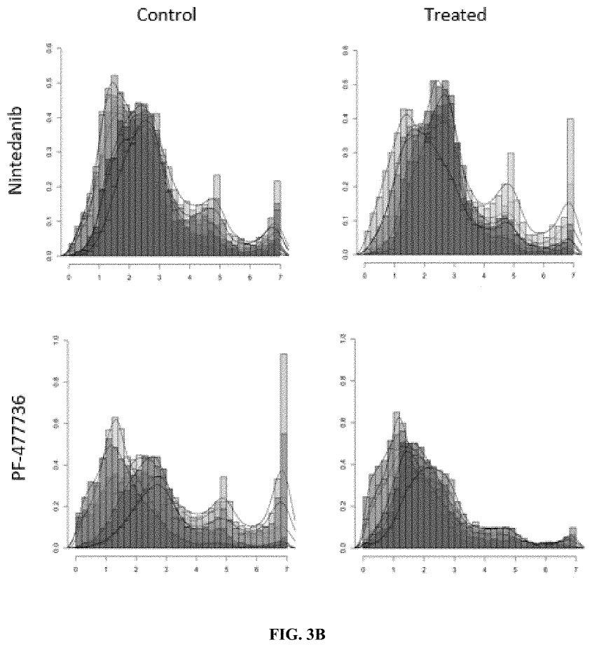

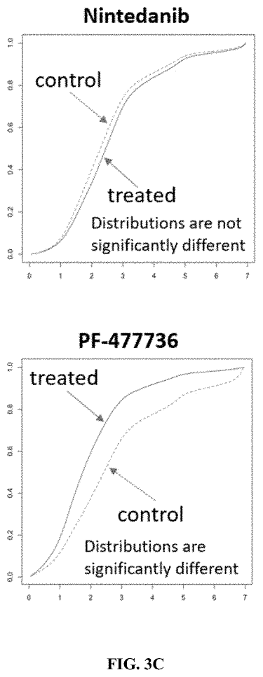

At the end of the study, lungs were processed using standard histological methods for Masson's Trichrome staining and were assigned Ashcroft scores by a blinded pathologist. As shown in FIG. 3A, PF-477736 and nintedanib improved histopathology scores in the mouse model of pulmonary fibrosis.

Whole lung imaging was performed using a modified deep learning algorithm to calculate an average histogram (FIG. 3B) and empirical cumulative density function (ECDF, FIG. 3C) for each arm. The X axes represent severity of pathology, and the Y axes represent frequency (histograms) or cumulative frequency (ECDF). The data demonstrate that PF-477736 treatment resulted in a greater reduction in pathology severity compared to nintedanib. For example, the shift of the ECDF curve for PF-477736 indicates a significantly different distribution, with lower pathology for PF-477736-treated lungs.

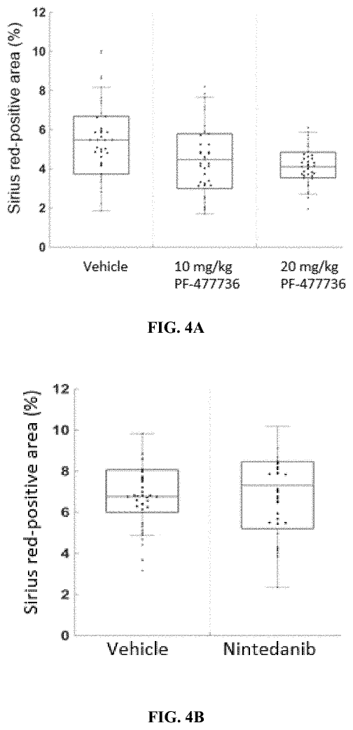

Histological sections were also stained using Sirius red and assessed for collagen deposition (%), collagen fiber count, collagen fiber density, and collagen fiber alignment in a protocol modified from Bredfeldt et al. 2014, “Computational segmentation of collagen fibers from second-harmonic generation images of breast cancer.” Journal of biomedical optics 19.1. As shown in FIG. 4A, FIG. 4B, and Table 8, PF-477736 reduced disease burden by all of these parameters in a dose-dependent manner, while treatment with nintedanib did not.

These data demonstrate that Chk1 inhibition exhibits therapeutic effects in an in vivo model of pulmonary fibrosis.

TABLE 8
Table 8: changes in collagen deposition, collagen fiber count, collagen
fiber density, and collagen fiber alignment for histological sections
from mice treated with PF-477736 or Nintedanib. The “multi-dose”
data include mice from both the 10 mg/kg and the 20 mg/kg PF-477736-
treated groups, and the associated p values demonstrate a significant
dose response. All values are relative vs vehicle.

| | Collagen | | | |
|---|---|---|---|---|
| | deposition (% | Fiber | Fiber | Fiber |
| Treatment | pixel count) | Count | Density | Alignment |
| PF-477736 | −1.64 | −66.3 | −0.66 | 0.08 |
| (20 mg/kg) | [p = 0.0029] | [p = 0.027] | [p = 0.045] | [p = 0.021] |
| PF-477736 | −1.27 | NS | NS | NS |
| (10 mg/kg) | [p = 0.12] | | | |
| PF-477736 | −1.63 | −67.7 | −0.69 | 0.08 |
| Multi-dose | [p = .0167] | [p = 0.013] | [p = 0.053] | [p = 0.026] |
| Nintedanib | +0.31 | +20 | +0.40 | −0.03 |
| | [p = 0.70] | [p = 0.41] | [p = 0.37] | [p = 0.38] |

### Example 5: Chk1 Activity is Found in IPF-Specific Populations of Epithelial Cells, Fibroblasts and Macrophages

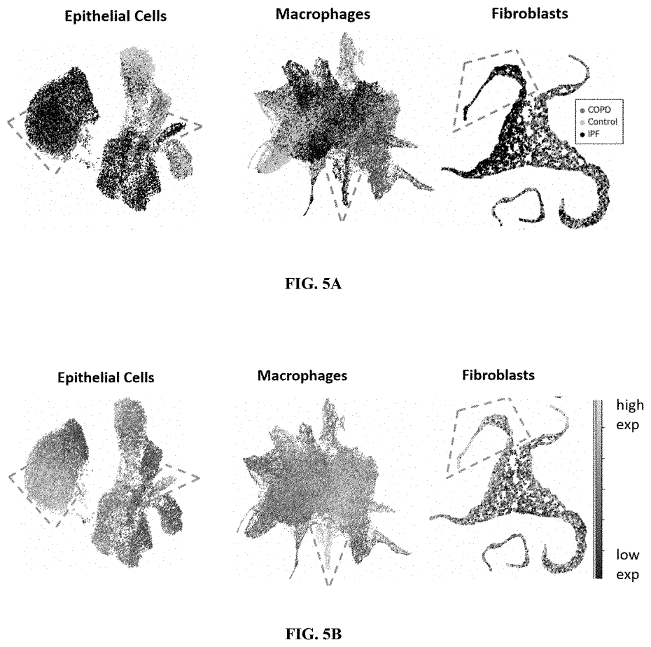

Single cell RNAseq was performed on 79 donor lungs, including 32 IPF, 29 healthy control, and 18 COPD lungs. Samples were dissociated and single cell RNA sequencing was performed. Epithelial cells, macrophages, and fibroblasts were identified based on expression of characteristic markers. Uniform Manifold Approximation and Projection (UMAP) clustering analysis allowed identification of IPF-specific epithelial, macrophage, and fibroblast cell populations, indicated by the dashed boxes in FIG. 5A and FIG. 5B.

To evaluate Chk1 activity, an expression signature of 100 genes was utilized. The 100 genes were chosen based on the observation that their expression correlates with Chk1 kinase activity. The signature of 100 Chk1-correlated genes was constructed using the ARCH4 database and applied to these data to infer which cell populations demonstrated the strongest activity. Transcriptional data underwent manifold-based dimensionality reduction and visualization using UMAP plots to delineate subpopulations stratify gene expression. Expression of the 100 genes was quantified at the single cell level. As shown in FIG. 5B, IPF-specific epithelial cells, macrophages, and fibroblasts exhibit enhanced Chk1 activity.

These data show that Chk1 activity is increased in multiple cell populations in IPF lungs compared to healthy control lungs and COPD lungs.

### Example 6: Combining a Chk1 Inhibitor with Nintedanib Results in an Additive Effect

Human lung fibroblasts from three donors were seeded in 96-well plates and treated with 1.25 ng/mL TGFb to induce differentiation to a myofibroblast phenotype (characterized by the induction of alpha smooth muscle actin—aSMA). A Chk1 inhibitor PF-477736, or standard-of-care compounds in IPF (nintedanib or pirfenidone) were dosed as indicated in an 8-point concentration curve 1 hr prior to TGFb treatment. Alpha-SMA and DAPI staining were assessed after 72 hours using high content analysis for percent inhibition of aSMA induction and % viable cells. CRC=concentration response curve. The first noted compound was dosed in an 8-point concentration curve, while the second noted compound was dosed at the pre-determined IC20 in this assay.

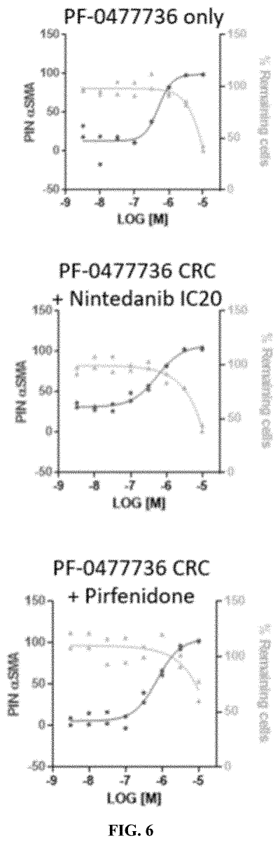

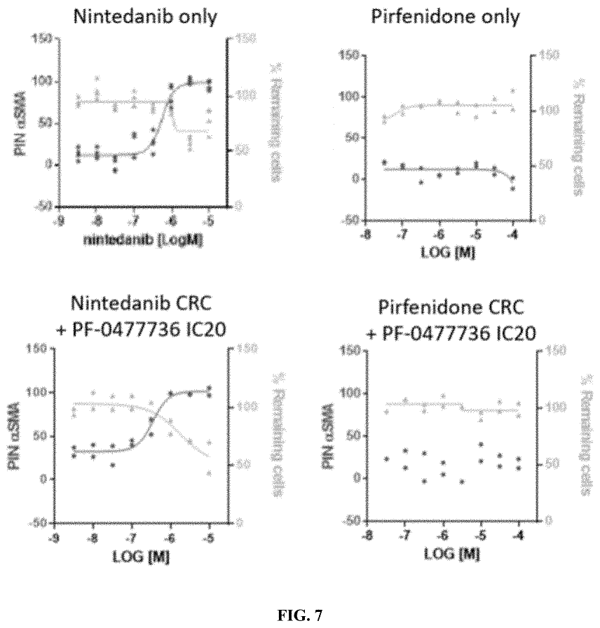

For each condition, percent inhibition (PIN) of aSMA induction was calculated (circles, and left y-axis), and the percent of viable cells was calculated (triangles, right y-axis). The concentration of Chk1 inhibitor required for 50% inhibition of fibroblast to myofibroblast differentiation was lower when co-administered with nintedanib (FIG. 6). The concentration of nintedanib required for 50% inhibition of fibroblast to myofibroblast differentiation was lower when co-administered with a Chk1 inhibitor (FIG. 7).

The combination of the Chk1 inhibitor with nintedanib or pirfenidone did not result in increased cell death (FIG. 6 and FIG. 7).

These data demonstrate that combining Chk1 inhibitors of the disclosure with other treatments (e.g., nintedanib) can result in additive effects that are useful in the treatment of idiopathic pulmonary fibrosis, without high toxicity.

### Example 7: High Chk1 Activity Correlates with Expression of Senescence-Associated Secreted Proteins in Epithelial Cells

This example demonstrates that epithelial cells from idiopathic pulmonary fibrosis patients with high levels of Chk1 activity also have express high levels of senescence-associated genes/senescence-associated secretory proteins, indicating that Chk1 inhibition can be useful for the treatment of IPF via an impact on senescence-associated gene expression.

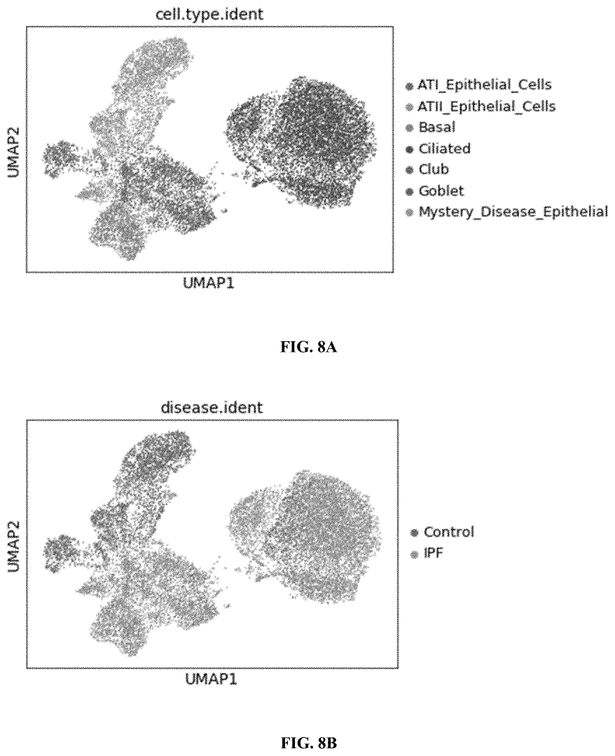

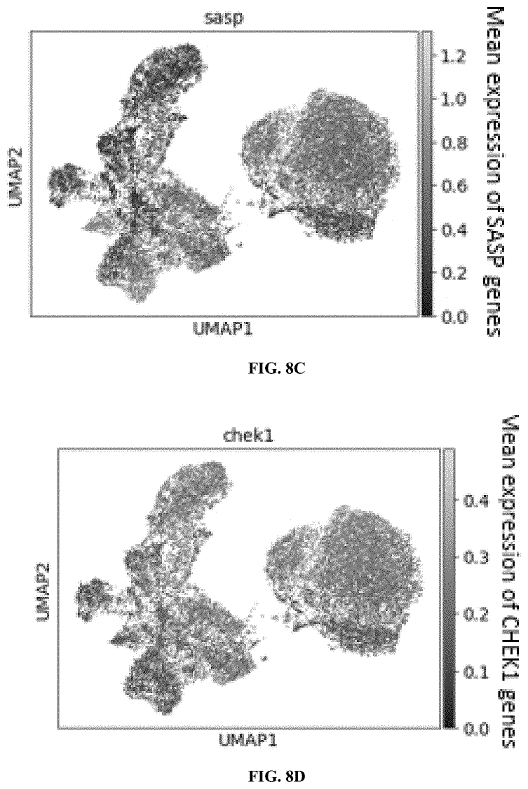

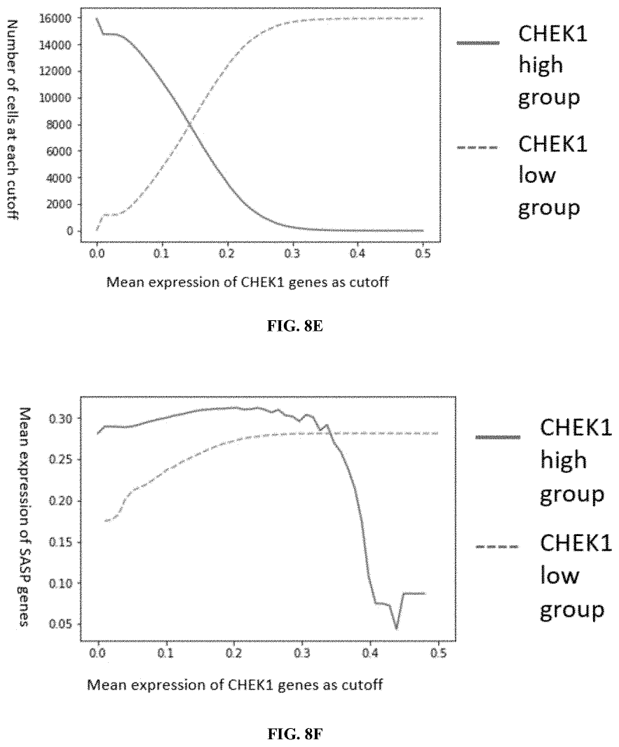

Single cell RNAseq was performed on 79 donor lungs, including 32 IPF, 29 healthy control, and 18 COPD lungs. Samples were dissociated and single cell RNA sequencing was performed. Various types of epithelial cells were identified in the scRNASeq dataset based on gene expression profiles, including alveolar type I (AT-I) epithelial cells, alveolar type II (AT-II) epithelial cells, basal epithelial cells, ciliated epithelial cells, club epithelial cells, goblet cells, and an IPF-associated epithelial cell subset (FIG. 8A). Cells identified as being from IPF patients and healthy controls are shown in FIG. 8B.

Expression of senescence-associated genes was evaluated, and the results are summarized in FIG. 8C. The senescence associated genes were those identified in Rana et al (2019)., PAI-1 Regulation of TGF-β1-induced ATII Cell Senescence, SASP Secretion, and SASP-mediated Activation of Alveolar Macrophages, American journal of respiratory cell and molecular biology.

A signature of 100 Chk1-correlated genes was used to infer which cell populations demonstrated the strongest Chk1 activity. The 100 genes were chosen based on the observation that their expression correlates with Chk1 kinase activity. The signature of 100 Chk1-correlated genes was constructed using the ARCH4 database and applied to these data to infer which cell populations demonstrated the strongest Chk1 activity. Transcriptional data underwent manifold-based dimensionality reduction and visualization using UMAP plots to delineate subpopulations stratify gene expression. Expression of the 100 genes was quantified at the single cell level, as summarized in FIG. 8D.

FIGS. 8A-D show that Chk1 high Chk1 activity correlates with expression of senescence-associated secreted proteins in epithelial cells from idiopathic pulmonary fibrosis patients, including, for example, in ciliated, club, basal, goblet, and IPF-specific epithelial cell subsets. The higher expression of senescence associated genes/senescence associated secretory proteins in cells with high Chk1 activity is further demonstrated in FIG. 8E and FIG. 8F, in which mean expressions of senescence genes in the Chk1-low and Chk1-high groups are plotted against thresholds of Chk1 expression. FIG. 8E shows that a threshold of approximately 0.1 to 0.2 is appropriate for differentiating between Chk1 high and Chk1 low cells. FIG. 8F shows that the Chk1 high group expresses higher mean levels of senescence-associated secretory proteins when a threshold of 0.1 to 0.2 is applied, and that in general, cells with high Chk1 activity have higher expression of senescence associated genes.

These data show that epithelial cells from idiopathic pulmonary fibrosis patients with high levels of Chk1 activity also have express high levels of senescence-associated genes/senescence-associated secretory proteins, indicating that Chk1 inhibition can impact senescence-associated gene expression, which can be useful for the treatment of IPF.

### Example 8: Chk1 Inhibition Decreases Histopathology and Collagen Deposition in a Mouse Model of Pulmonary Fibrosis

To induce fibrosis, 6-8 week old C57BL6 mice are dosed with 3 mg/kg bleomycin hydrochloride in a volume of 50 μL per animal via intratracheal (i.t.) administration with a microsprayer. One week following administration of bleomycin, mice are treated with compound or vehicle once daily from day 7 to day 20. A Chk1 inhibitor of the disclosure (for example, AB-IsoG (isogranulatimide); AZD-7762; CCT-244747; CHK1-A; GNE-900; MK-8776; PF-477736; rabusertib; GDC-0425; GDC-0575; SAR 020106; V-158411; XL-844; ARRY 575; CASC-578; LY-2880070; CCT-245737; CCT-241533; prexasertib; VER-250840; or BML-277) is administered (e.g., i.p. in a suitable vehicle, e.g., 50 nM sodium acetate buffer and 4% dextrose, pH 4). Nintedanib is administered orally at 100 mg/kg in a vehicle of 1% methylcellulose as a control. n=10 mice per group.

At the end of the study, lungs are processed using standard histological methods for Masson's Trichrome staining and are assigned Ashcroft scores by a blinded pathologist. The Chk1 inhibitor improves histopathology scores in the mouse model of pulmonary fibrosis.

Whole lung imaging is performed using a modified deep learning algorithm to calculate an average histogram and empirical cumulative density function (ECDF) for each arm of the study. The data demonstrate that Chk1 inhibitor treatment results in a greater reduction in pathology severity compared to nintedanib.

Histological sections are also stained using Sirius red and assessed for collagen deposition (%), collagen fiber count, collagen fiber density, and collagen fiber alignment in a protocol modified from Bredfeldt et al. 2014, “Computational segmentation of collagen fibers from second-harmonic generation images of breast cancer.” Journal of biomedical optics 19.1. Chk1 inhibition reduces disease burden by one or more of these parameters in a dose-dependent manner.

The terminology and description used herein is for the purpose of describing particular embodiments only and is not intended to limit the scope of the invention.

1-62. (canceled)

63. A method of treating pulmonary fibrosis in a subject in need thereof, comprising administering to the subject a therapeutically-effective amount of a pharmaceutical composition, wherein the pharmaceutical composition comprises a Chk1 inhibitor, wherein upon the administering, the Chk1 inhibitor reduces a level of fibroblast to myofibroblast differentiation in the subject by at least about 5% relative to a control.

64. The method of claim 63, wherein the pulmonary fibrosis is idiopathic pulmonary fibrosis (IPF).

65. The method of claim 63, wherein the control is a level of fibroblast to myofibroblast differentiation in a control subject that was not administered the Chk1 inhibitor.

66. The method of claim 63, wherein the control is a level of fibroblast to myofibroblast differentiation in the subject prior to the administering the Chk1 inhibitor.

67. The method of claim 63, wherein the level of fibroblast to myofibroblast differentiation is as determined by contacting fibroblasts with TGF-β, staining the fibroblasts with a reagent that specifically stains alpha smooth muscle actin, and conducting high content analysis to determine percent inhibition of alpha smooth muscle actin induction.

68. The method of claim 63, wherein the Chk1 inhibitor is not a thiazole compound, a heterocyclic urea compound, a heterocyclic thiourea compound, or an anilinopiperazine compound.

69. The method of claim 63, wherein the Chk1 inhibitor is CCT-245737.

70. The method of claim 63, wherein the subject is a mammal.

71. The method of claim 63, wherein the subject is a human.

72. The method of claim 63, wherein the pharmaceutical composition is administered in a unit dosage form.

73. The method of claim 63, further comprising administering an additional therapeutic agent to the subject.

74. The method of claim 73, wherein the additional therapeutic agent comprises nintedanib.

75. The method of claim 73, wherein the additional therapeutic agent comprises pirfenidone.

76. The method of claim 73, wherein the additional therapeutic agent comprises an immunomodulatory agent.

77. The method of claim 63, wherein the pharmaceutical composition is administered via inhalation.

78. The method of claim 63, wherein the pharmaceutical composition is administered orally.

79. The method of claim 63, wherein the pharmaceutical composition is administered parenterally.

80. The method of claim 63, wherein the therapeutically-effective amount is from about 0.1 μg/kg to about 100 mg/kg.

81. A method of reducing differentiation of a fibroblast into a myofibroblast, comprising contacting a population of cells with a Chk1 inhibitor, wherein the population of cells comprises the fibroblast, wherein upon contacting the population of cells with the Chk1 inhibitor, the fibroblast exhibits an expression level of alpha smooth muscle actin that is at least about 5% lower than an expression level of the alpha smooth muscle actin by a fibroblast that was not contacted with the Chk1 inhibitor.

82. A method of reducing collagen deposition, comprising contacting a tissue with a Chk1 inhibitor, wherein upon contacting the tissue with the Chk1 inhibitor, the tissue exhibits an at least about 5% lower level of an indicator of collagen deposition relative to a tissue that was not contacted with the Chk1 inhibitor.
